(19) United States
(12) Patent Application Publication
(10) Pub. No.: US 2003/0044814 A1
(43) Pub. Date: Mar. 6, 2003

(54) PRC17: an amplified cancer gene

(75) Inventors: Jing Li, Flushing, NY (US); Scott Powers, Greenlawn, NY (US); Phil Xiang, San Francisco, CA (US); Yue Peng, South Setauket, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tularik Inc., South San Francisco, CA (US)

(21) Appl. No.: 10/071,838

(22) Filed: Feb. 8, 2002

Related U.S. Application Data
(60) Provisional application No. 60/267,615, filed on Feb. 8, 2001.

(51) Int. Cl. C12Q 1/68; G01N 33/574; C12P 19/34

(52) U.S. Cl. 435/6; 435/7.23; 435/91.2

(57) ABSTRACT

The present invention provides nucleic acid and protein sequences for a novel protein, PRC17, and methods, reagents, and kits for diagnosing and treating cancer in a mammal, e.g., a human. This invention is based upon the discovery that the novel PRC17 is overexpressed and/or amplified in cancer. Methods to detect cancer or a propensity to develop cancer, to monitor the efficacy of a cancer treatment, and to treat cancer, by inhibiting the expression and/or activity of PRC17 in a cancer cell are included.

### CROSS REFERENCE TO RELATED APPLICATIONS

The current application claims benefit of priority to U.S. Application No. 60/267,615, filed Feb. 8, 2001, which is herein incorporated by reference.

### FIELD OF THE INVENTION

The present invention provides isolated nucleic acids encoding a novel protein, PRC17. In particular, the present invention provides reagents and methods for diagnosing and treating cancers that are associated with amplification or overexpression of PRC17 nucleic acids and polypeptides.

### BACKGROUND OF THE INVENTION

Cancer is a genetic disease of single cell origin caused by the accumulation of inherited and acquired mutations in specific cancer genes, which have normal cellular functions, but when mutated or present at abnormally high levels contribute to cancer. One type of mutation is gene amplification or overexpression, i.e., where a specific chromosomal region (including the cancer gene) has undergone a relative increase in DNA copy number, such that more copies of the cancer gene are present, or where the level of expression of a gene is increased, such that a correspondingly higher amount of mRNA and protein is produced, causing deleterious effects. Gene amplification is one of the primary genetic alterations in solid tumors, and most of the chromosomal regions that undergo amplification are not well characterized and do not harbor known oncogenes (Knuutila et al., (1998) Am. J. Pathol., 152:1107-23; Knuutila et al., (1998) Cancer Genet. Cytogenet., 100:25-30). Discovery of these amplified cancer genes will provide novel targets for diagnostic and therapeutic applications.

### SUMMARY OF THE INVENTION

The present invention provides isolated nucleic acids encoding a novel protein, PRC17. In particular, the invention provides PRC17 polypeptide and polynucleotide sequences, which are amplified or overexpressed in cancer cells. Accordingly, the reagents and methods of the invention can be used to detect cancer or a propensity to develop cancer, to monitor the efficacy of a cancer treatment, to identify modulators of PRC17 activity, particularly inhibitors of PRC17, and to treat diseases or conditions associated with PRC17 activity or expression, particularly cancer, e.g., by inhibiting the expression and/or activity of PRC17 in a cancer cell.

In one aspect, the invention provides an isolated nucleic acid encoding a PRC17 polypeptide, wherein the PRC17 polypeptide comprises at least 85% amino acid identity to an amino acid sequence of SEQ ID NO:2 or at least 70% identity to an amino acid sequence of SEQ ID NO:4 or SEQ ID NO:6. In one embodiment, the polypeptides comprises an amino acid sequence of SEQ ID NO:2, SEQ ID NO:4 or SEQ ID NO:6. In another embodiment, the nucleic acid encodes a polypeptide that specifically binds to polyclonal antibodies generated against a polypeptide having an amino acid sequence of SEQ ID NO:2, SEQ ID NO:4 or SEQ ID NO:6. Often, the nucleic acid hybridizes under stringent or, alternatively, moderately stringent hybridization conditions to a nucleic acid comprising a nucleotide sequence of SEQ ID NO: l, SEQ ID NO:3 or SEQ ID NO:5. In one embodiment the nucleic acid comprises a nucleotide sequence of SEQ ID NO:1, SEQ ID NO:3 or SEQ ID NO:5.

In another aspect, the invention provides an expression vector comprising a nucleic acid encoding a PRC17 polypeptide or an isolated cell comprising the expression vector.

Other embodiments include an isolated polypeptide or subfragment that comprises at least 85% amino acid identity to an amino acid sequence of SEQ ID NO:2 or at least 70% identity to an amino acid sequence of SEQ ID NO:4 or SEQ ID NO:6., often the polypeptide comprises the amino acid sequence of SEQ ID NO:2, SEQ ID NO:4 or SEQ ID NO:6.

In another aspect, the present invention provides a method of detecting the presence of tumor tissue in a biological sample from a mammal, the method comprising providing the biological sample from the mammal and detecting an increase in copy number of a gene encoding PRC17 polypeptide in the biological sample, thereby detecting the presence of tumor tissue in the biological sample. In one embodiment, the detecting step comprises contacting the gene with a probe that selectively hybridizes to the gene under conditions in which the probe selectively hybridizes to the gene to form a stable hybridization complex and detecting the hybridization complex. Often, the contacting step includes a step of amplifying the gene in an amplification reaction. In one embodiment, the amplification reaction is a polymerase chain reaction.

In a further embodiment, the tumor tissue is from an epithelial tumor, often a prostate tumor, a colon tumor, an ovarian tumor or a breast tumor. In a presently preferred embodiment, the epithelial tumor is a prostate tumor. In other embodiments, the mammal is a human and the biological sample is a tissue biopsy, often a prostate tissue biopsy.

In another aspect, the invention provides a method of monitoring the efficacy of a therapeutic treatment of a tumor, the method comprising providing a biological sample from a mammal undergoing the therapeutic treatment, and detecting a level of a PRC17 polypeptide in the biological sample compared to a level in a biological sample from the mammal prior to, or earlier in, the therapeutic treatment, thereby monitoring the efficacy of the therapy. In one embodiment, the tumor is an epithelial tumor, often a prostate, colon, ovarian or breast tumor. In a presently preferred embodiment, the epithelial tumor is a prostate tumor. In another embodiment the mammal is a human.

The invention also provides a method of identifying a compound that inhibits the activity of a PRC17 polypeptide, the method comprising contacting the PRC17 polypeptide with a compound and detecting a decrease in the activity of the PRC17 polypeptide. In one embodiment, the polypeptide is linked to a solid phase. In another embodiment, the PRC17 polypeptide is expressed in a cell. Additionally, the PRC17 polypeptide can be amplified in the cell compared to normal.

In another aspect, the invention provides a method of treating a cancer that is associated witih PRC17 overexpression, the method comprising the step of contacting a tumor cell with a therapeutically effective amount of an inhibitor of PRC17. In one embodiment the tumor cell is an epithelial tumor cell, often a prostate, ovarian, colon or breast tumor, preferably a prostate tumor. In another embodiment, the inhibitor is identified using a method of identifying a compound that inhibits the activity of PRC17, the method comprising contacting the PRC17 polypeptide with a compound and detecting a decrease in the activity of the PRC17 polypeptide. In one embodiment, the inhibitor is an antibody. Alternatively, the inhibitor is an antisense polynucleotide.

In another aspect, the present invention provides a method of detecting tumor tissue in a biological sample from a mammal, the method comprising providing the biological sample from the mammal, and detecting overexpression of a PRC17 polypeptide in the biological sample, thereby detecting tumor tissue in the biological sample.

The method includes an embodiment in which a PRC17 polypeptide is detected using an antibody that selectively binds to PRC17. Often, the amount of PRC17 polypeptide is quantified by immunoassay. In another embodiment, detecting the overexpression of a PRC17 polypeptide comprises detecting the activity of the PRC17 polypeptide.

In an alternative embodiment, detecting overexpression of a PRC17 polypeptide comprises detecting an mRNA that encodes the PRC17 polypeptide. Often, the mRNA is detected using an amplification reaction.

### BRIEF DESCRIPTION OF THE DRAWINGS

FIG. 1 illustrates the epicenter mapping of the 17q12 amplicon. DNA copy number was evaluated using Q-PCR. Fold amplification was plotted against the fluorogenic Taqman probes' physical positions. The probes, which were designed based on Ests or BAC sequences, were ordered on the basis of their physical presence on the BAC contig. The regional BAC contig was assembled based on the sequence homology and physical presence of BAC ends. The assembly of BAC sequences that we used for this analysis agrees with published assembly from HGREP (http://hgrep.ims.u-tokyo.acjp).

FIG. 2 illustrates detection of increased amounts of endogenous PRC17 protein in metastatic prostate tumor samples. PRC17 protein levels in 3T3 cells stably transfected with PRC17 and in metastic prostate tumor samples (WA5-3, WA5-4, WA20-45, and WA12-2) were measured by Western blot.

FIG. 3 shows the interaction of PRC17 with Rab5 and shows that PRC17 stimulates GTP hydrolysis on Rab5. (3A) Interaction between PRC17 and Rab5. Whole cell lysate from indicated cell lines were immunoprecipitated with anti-Rab5 antibody, and the immunocomplexes were analysed by Western blot using anti-RPC17 antibody. (3B) Purification of PRC17 and Rab5 from E. Coli: Coomassie blue staining of purified recombinant proteins. Lane 1: wild type PRC17, Lane 2: mutant PRC17, Lane 3: Rab5. (3C) GTP hydrolysis assay. Rab5 (30 ng) was loaded with [γ-32P]GTP and incubated with BSA, or purified wild type or mutant PRC17 (200 ng). GAP activity was expressed as the percentage of non-hydrolysed [γ-32P]GTP that remained bound to the filters, relative to the radioactivity at time 0. Values are the mean of three independent experiments.

FIG. 4 shows the oncogenic properties of PRC17. (4A) Cell proliferation assay. Cell growth rate is expressed as absorbance at 490 nm. 3T3-pLPC is in white; 3T3-PRC17 wild type is in black; 3T3 PRC17 mutant is in gray. Two independent experiments (n=8 for each experiment). (4B) 3T3 stable transfectants with vector alone (pLPC), or PRC17 wild type or PRC17 mutant (5×106 cells) were injected into nude mice (n=5). Tumor formation was monitored. The tumors were measured each week at their longest points using a caliper.

FIG. 5 illustrates the amino acid comparison between PRC17 and TRE-2/USP6, a known oncogene. PRC17 shares 81% identity on the protein level and 88% identity on the DNA level to TRE-2/USP6.

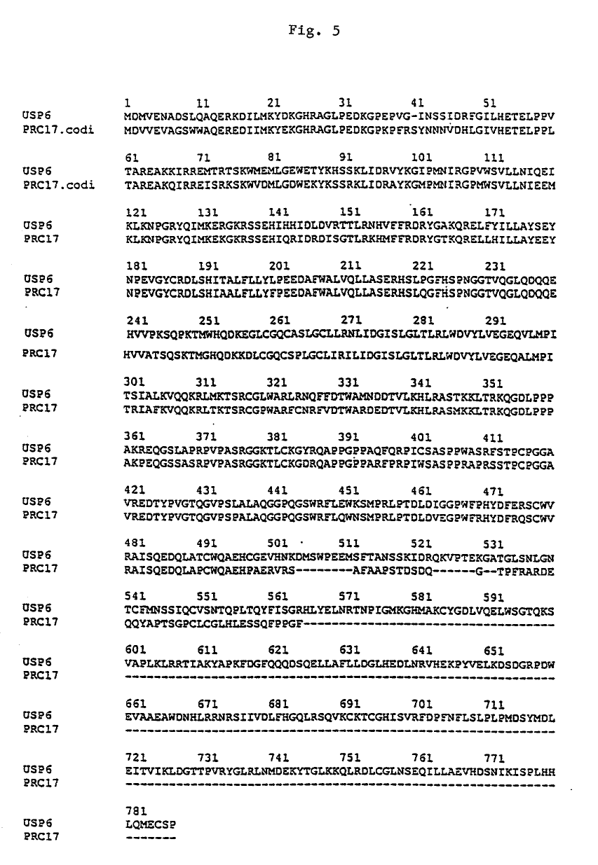

### DETAILED DESCRIPTION OF THE INVENTION AND PREFERRED EMBODIMENTS

I. Introduction

The present invention provides isolated nucleic acid and amino acid sequences encoding PRC17 and methods of production of PRC17. The sequences can be used for the specific detection of cells expressing PRC17, for the identification of molecules that associate with and/or modulate the activity of PRC17, or for the diagnosis of cancer or other diseases or conditions associated with PRC17 amplification or PRC17 activity or expression. In one aspect, the invention is based upon the discovery that the PRC17 gene is overexpressed and/or amplified in cancer cells, particularly prostate cancer cells. Accordingly, the present methods can be used to detect cancer or a propensity to develop cancer, to monitor the efficacy of a cancer treatment, and to treat cancer, e.g., by inhibiting the expression and/or activity of PRC17 in a cancer cell.

PRC17 is homologous to RTE2/USP6 and contains a conserved domain that is predicted to be the catalytic core of a GTPase-activating protein (GAP) directed towards Rab/Ypt type GTP-binding proteins. The predicted hyperactivity of amplified PRC17 in prostate tumors could be inhibited by a small molecular inhibitor of PRC17 GAP activity. The ubiquitin carboxyl-terminal hydrolases domain, UCH-1, which is present in TRE2/USP6 is absent in PRC17, but it has never been shown whether that function of TRE2/USP6 is critical for its transforming oncogenic activity.

The present invention provides isolated nucleic acid and amino acid sequences encoding PRC17 and methods of production of PRC17. The invention also provides methods of screening for modulators, e.g., activators, inhibitors, stimulators, enhancers, etc. of PRC17 nucleic acids and proteins. Such modulators can affect PRC17 activity, e.g., by modulating PRC17 transcription, translation, mRNA or protein stability; by altering the interaction of PRC17 with the plasma membrane, or other molecules; or by affecting PRC17 protein activity. In one embodiment, compounds are screened, e.g., using high throughput screening (HTS), to identify those compounds that can bind to and/or modulate the activity of an isolated PRC17 polypeptide or fragment thereof. In another embodiment, PRC17 proteins are recombinantly expressed in cells, and the modulation of PRC17 is assayed by using any measure of PRC17 function, such as association with the cell membrane or binding partners.

In numerous embodiments, a PRC17 polynucleotide or polypeptide is introduced into a cell, in vivo or ex vivo, and the PRC17 activity in the cell is thereby modulated. For example, a polynucleotide encoding a full length PRC17 polypeptide can be introduced into a population of cells, thereby modulating the electrophysiological properties of the cells.

In certain embodiments, monoclonal or polyclonal antibodies directed to PRC17, or subfragment or domain of PRC17, will be administered to a mammal to inhibit the activity of PRC17 in cells. Such embodiments are useful, e.g., in the treatment of a disease or disorder associated with PRC17 activity.

The present invention also provides methods for detecting PRC17 nucleic acid and protein expression. PRC17 polypeptides can also be used to generate monoclonal and polyclonal antibodies useful for the detection of PRC17-expressing cells or for the amelioration of PRC17 activity. Cells that express PRC17 can also be identified using techniques such as reverse transcription and amplification of mRNA, isolation of total RNA or poly A⁺ RNA, northern blotting, dot blotting, in situ hybridization, RNase protection, S1 digestion, probing DNA microchip arrays, western blots, and the like.

Nucleotide and amino acid sequence information for PRC17 are also used to construct models of PRC17 proteins. These models are subsequently used to identify compounds that can activate or inhibit PRC17 proteins. Such compounds that modulate the activity of PRC17 genes or proteins can be used to investigate the physiological role of PRC17 genes.

The present invention also provides assays, preferably high throughput screening (HTS) assays, to identify compounds or other molecules that interact with and/or modulate PRC17. In certain assays, a particular domain of PRC17 is used, e.g., a conserved domain that is thought to be the catalytic core of a GTPase-activating protein (GAP).

The present invention also provides methods to diagnose or treat diseases or conditions associated with PRC17 activity. For example, PRC17 activity and/or expression can be altered in cells of a patient with a PRC17-associated disease. In particular, the invention provides for methods of diagnosing or treating cancer.

Methods of the invention directed to diagnosing or treating cancer typically involve detecting the presence of PRC17 in a biological sample taken from a mammal. In certain embodiments, a level of PRC17 in a biological sample will be compared with a control sample taken from a cancer-free mammal or, preferably, with a value expected for a sample taken from a cancer-free mammal. A control sample can also be obtained from normal tissue from the same mammal that is suspected of having cancer.

The ability to detect cancer cells by virtue of an increased level of PRC17 is useful for any of a large number of applications. For example, an increased level of PRC17 in cells of a mammal can be used, alone or in combination with other diagnostic methods, to diagnose cancer in the mammal or to determine the propensity of a mammal to develop cancer over time. The detection of PRC17 can also be used to monitor the efficacy of a cancer treatment. For example, a level of a PRC17 polypeptide or polynucleotide after an anti-cancer treatment is compared to the level in the mammal before the treatment. A decrease in the level of the PRC17 polypeptide or polynucleotide after the treatment indicates efficacious treatment.

An increased level or diagnostic presence of PRC17 can also be used to influence the choice of anti-cancer treatment in a mammal, where, for example, the level of PRC17 increase directly correlates with the aggressiveness of the anti-cancer therapy. For example, an increased level of PRC17 in tumor cells can indicate that the use of an agent that decreases proliferation would be effective in treating the tumor.

In addition, the ability to detect cancer cells can be used to monitor the number or location of cancer cells in a patient, in vitro or in vivo, for example, to monitor the progression of the cancer over time. In addition, the level or presence or absence of PRC17 can be statistically correlated with the efficacy of particular anti-cancer therapies or with observed prognostic outcomes, thereby allowing for the development of databases based on a statistically-based prognosis, or a selection of the most efficacious treatment, can be made in view of a particular level or diagnostic presence of PRC17.

The present invention also provides methods for treating cancer. In certain embodiments, the proliferation of a cell with an elevated level of PRC17 polynucleotides, polypeptides, or polypeptide activity is inhibited. In other embodiments, PRC17 expression is not elevated compared to normal, but PRC17 activity, for example, functions at the cell surface membrane, can be blocked or inhibited to prevent tumor cell growth, migration, or metastasis. Proliferation and/or migration is decreased by, for example, contacting the cell with an inhibitor of PRC17 transcription or translation, or an inhibitor of the activity of a PRC17 polypeptide. Such inhibitors include, but are not limited to, antisense polynucleotides, ribozymes, antibodies, dominant negative PRC17 polypeptides, and small molecule inhibitors of PRC17 activity.

The present methods can be used to diagnose, determine the prognosis for, or treat, any of a number of types of cancers. In preferred embodiments, the cancer is an epithelial cancer, e.g., prostate, lung, breast, colon, kidney, stomach, bladder, or ovarian cancer, or any cancer of the gastrointestinal tract. In a presently preferred embodiment, the cancer is prostate cancer.

The diagnostic methods of this invention can be used in animals including, for example, primates, canines, felines, murines, bovines, equines, ovines, porcines, lagomorphs, etc, as well as in humans. In a preferred embodiment, the mammal is a human.

Kits are also provided for carrying out the herein-disclosed diagnostic and therapeutic methods.

II. Definitions

As used herein, the following terms have the meanings ascribed to them unless specified otherwise.

A “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

The phrase “detecting a cancer” refers to the ascertainment of the presence or absence of cancer in an animal. “Detecting a cancer” can also refer to obtaining indirect evidence regarding the likelihood of the presence of cancerous cells in the animal. Detecting a cancer can be accomplished using the methods of this invention alone, in combination with other methods, or in light of other information regarding the state of health of the animal.

The term “PRC17” refers to PRC17 nucleic acid and polypeptide polymorphic variants, alleles, mutants, and interspecies homologs that: (1) have an amino acid sequence that has greater than about 60% amino acid sequence identity, preferably 65%, 70%, 75%, 80%, 85%, 90%, preferably 86%, 87%, 88%, 89%, 90%, 91%, 92%, 93%, 94%, 95%, 96%, 97%, 98% or 99% or greater amino acid sequence identity, preferably over a region of over a region of at least about 50, 100, 200, 500, 1000, or more amino acids, to a PRC17 sequence of SEQ ID NO:2; (2) bind to antibodies, e.g., polyclonal antibodies, raised against an immunogen comprising an amino acid sequence of SEQ ID NO:2, SEQ ID NO:4 or SEQ ID NO:6, and conservatively modified variants thereof; (3) specifically hybridize under stringent hybridization conditions to a PRC17 nucleic acid sequence of SEQ ID NO:1, SEQ ID NO:3 or SEQ ID NO:5 and conservatively modified variants thereof; (4) have a nucleic acid sequence that has greater than about 80%, preferably about 85% or 90%, preferably greater than about 96%, 97%, 98%, 99%, or higher nucleotide sequence identity, preferably over a region of over a region of at least about 50, 100, 200, 500, 1000, or more nucleotides, to SEQ ID NO:1, SEQ ID NO:3 or SEQ ID NO:5; or (5) are amplified by primers that specifically hybridize under stringent hybridization conditions to the same sequence as a primer set selected from the group consisting of SEQ ID NOs:7 and 8; and SEQ ID NOs:10 and 11. A PRC17 polynucleotide or polypeptide sequence is typically from a mammal including, but not limited to, human, rat, mouse, hamster, cow, pig, horse, sheep, or any mammal. A “PRC17 polynucleotide” and a “PRC17 polypeptide” are both either naturally occurring or recombinant. The human PRC17 gene is located at chromosome 17q11-12 based on the Human Genome Project draft sequence data.

A “full length” PRC17 protein or nucleic acid refers to a PRC17 polypeptide or polynucleotide sequence, or a variant thereof, that contains all of the elements normally contained in one or more naturally occurring, wild type PRC17 polynucleotide or polypeptide sequences.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques. In some embodiments, the nucleotide sequences that encode the enzymes are preferably optimized for expression in a particular host cell (e.g., yeast, mammalian, plant, fungal, and the like) used to produce the enzymes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

| | |
|---|---|
| 1) | Alanine (A), Glycine (G); |
| 2) | Aspartic acid (D), Glutamic acid (E); |
| 3) | Asparagine (N), Glutamine (Q); |
| 4) | Arginine (R), Lysine (K); |
| 5) | Isoleucine (I), Leucine (L), Methionine (M), Valine (V); |
| 6) | Phenylalanine (F), Tyrosine (Y), Tryptophan (W); |
| 7) | Serine (S), Threonine (T); and |
| 8) | Cysteine (C), Methionine (M) |

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

“Biological sample,” as used herein, refers to a sample of cells, biological tissue or fluid that contains one or more PRC17 nucleic acids encoding one or more PRC17 proteins. Most often, the sample has been removed from an animal, but the term “biological sample” can also refer to cells or tissue analyzed in vivo, i.e., without removal from the animal. Typically, a “biological sample” will contain cells from the animal, but the term can also refer to noncellular biological material, such as noncellular fractions of blood, saliva, or urine, that can be used to measure PRC17 levels. Numerous types of biological samples can be used in the present invention, including, but not limited to, a tissue biopsy, a blood sample, a buccal scrape, a saliva sample, or a nipple discharge. Such samples include, but are not limited to, tissue isolated from humans, mice, and rats, in particular, breast and lung tissue as well as blood, lymphatic tissue, liver, brain, heart, spleen, testis, ovary, thymus, kidney, and embryonic tissues. Biological samples may also include sections of tissues such as frozen sections taken for histological purposes. A biological sample is typically obtained from a mammal such as rat, mouse, cow, dog, cat, guinea pig, or rabbit, and most preferably a primate such as a chimpanzee or a human.

“Providing a biological sample” means to obtain a biological sample for use in the methods described in this invention. Most often, this will be done by removing a sample of cells from an animal, but can also be accomplished by using previously isolated cells (e.g., isolated by another person, at another time, and/or for another purpose), or by performing the methods of the invention in vivo.

As used herein, a “tissue biopsy” refers to an amount of tissue removed from an animal for diagnostic analysis. In a patient with cancer, tissue may be removed from a tumor, allowing the analysis of cells within the tumor. “Tissue biopsy” can refer to any type of biopsy, such as needle biopsy, fine needle biopsy, surgical biopsy, etc.

A “control sample” refers to a sample of biological material representative of healthy, cancer-free animals. The level of PRC17 in a control sample is desirably typical of the general population of normal, cancer-free animals. This sample can be removed from an animal expressly for use in the methods described in this invention, or can be any biological material representative of normal, cancer-free animals. A control sample can also be obtained from normal tissue from the animal that has cancer or is suspected of having cancer. A control sample can also refer to an established level of PRC17, representative of the cancer-free population, that has been previously established based on measurements from normal, cancer-free animals. If a detection method is used that only detects PRC17 when a level higher than that typical of a normal, cancer-free animal is present, i.e., an immunohistochemical assay giving a simple positive or negative result, this is considered to be assessing the PRC17 level in comparison to the control level, as the control level is inherent in the assay.

The “level of PRC17 mRNA” in a biological sample refers to the amount of mRNA transcribed from a PRC17 gene that is present in a cell or a biological sample. The mRNA generally encodes a functional PRC17 protein, although mutations or microdeletions may be present that alter or eliminate the function of the encoded protein. A “level of PRC17 mRNA” need not be quantified, but can simply be detected, e.g., a subjective, visual detection by a human, with or without comparison to a level from a control sample or a level expected of a control sample.

The “level of PRC17 protein or polypeptide” in a biological sample refers to the amount of polypeptide translated from a PRC17 mRNA that is present in a cell or biological sample. The polypeptide may or may not have PRC17 protein activity. A “level of PRC17 protein” need not be quantified, but can simply be detected, e.g., a subjective, visual detection by a human, with or without comparison to a level from a control sample or a level expected of a control sample.

An “increased,” or “elevated,” level of PRC17 refers to a level of PRC17 polynucleotide, e.g., genomic DNA, or mRNA, or polypeptide, that, in comparison with a control level of PRC17, is detectably higher. The method of comparison can be statistical, using quantified values for the level of PRC17, or can be compared using nonstatistical means, such as by a visual, subjective assessment by a human.

For diagnostic and prognostic applications in cancer, a level of PRC17 polypeptide or polynucleotide that is “expected” in a control sample refers to a level that represents a typical, cancer-free sample, and from which an elevated, or diagnostic, presence of PRC17 polypeptide or polynucleotide can be distinguished. Preferably, an “expected” level will be controlled for such factors as the age, sex, medical history, etc. of the mammal, as well as for the particular biological sample being tested.

The phrase “functional effects” in the context of assays for testing compounds that modulate PRC17 activity includes the determination of any parameter that is indirectly or directly under the influence of PRC17, e.g., a functional, physical, or chemical effect. These effects include gene amplification, or expression in cancer cells. “Functional effects” include in vitro, in vivo, and ex vivo activities.

By “determining the functional effect” is meant assaying for a compound that increases or decreases a parameter that is indirectly or directly under the influence of PRC17, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of PRC17; or binding assays, e.g., measuring the association of PRC17 with other proteins.

“Inhibitors” and “modulators” of PRC17 are used to refer to inhibitory or modulating molecules identified using in vitro and in vivo assays of PRC17, e.g., PRC17 expression in cell membranes. Inhibitors are compounds that, e.g., bind to, partially or totally block activity, decrease, prevent, delay activation, inactivate, desensitize, or down regulate the activity of PRC17, e.g., antagonists. Activators are compounds that, e.g., increase PRC17 activity, or increase PRC17 expression or stability. Modulators of PRC17 also include genetically modified versions of PRC17, e.g., versions with altered activity, as well as naturally occurring and synthetic ligands, antagonists, agonists, antibodies, small chemical molecules and the like. Assays for inhibitors and activators of PRC17 include, e.g., expressing PRC17 in vitro, in cells, or cell membranes, applying putative modulator compounds, and then determining the functional effects on PRC17 activity, as described above.

Samples or assays comprising PRC17 polypeptides that are treated with a potential activator, inhibitor, or modulator are compared to control samples without the inhibitor, activator, or modulator to examine the effect of the candidate compound. Control samples (untreated with the compound) are assigned a relative PRC17 activity value of 100%. Inhibition of a PRC17 polypeptide is achieved when the activity value relative to the control is about 80%, optionally about 50% or 25-0%. Activation of a PRC17 polypeptide is achieved when the activity value relative to the control is about 110%, optionally about 150%, optionally about 200-500%, or about 1000-3000% higher.

The terms “isolated”, “purified”, or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated PRC17 nucleic acid is separated from open reading frames that flank the PRC17 gene and encode proteins other than PRC17. The term “purified” denotes that a nucleic acid or protein gives rise to essentially one band in an electrophoretic gel. Particularly, it means that the nucleic acid or protein is at least 85% pure, optionally at least 95% pure, and optionally at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiralmethyl phosphonates, 2-0-methyl ribonucleotides, peptide-nucleic acids (PNAs).

Unless otherwise indicated, a particular nucleic acid sequence also implicitly encompasses conservatively modified variants thereof (e.g. degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “label” or a “detectable moiety” is a composition detectable by spectroscopic, photochemical, biochemical, immunochemical, or chemical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 86%, 87%, 88%, 89%, 90%, 91%, 92%, 93%, 94%, 95%, 96%, 97%, 98%, 99%, or higher identity over a specified region (e.g., SEQ ID NOS:1-6), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to the compliment of a test sequence. The definition also includes sequences that have deletions and/or additions, as well as those that have substitutions. As described below, the preferred algorithms can account for gaps and the like. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂ a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-PRC17” antibody is an antibody or antibody fragment that specifically binds a polypeptide encoded by a PRC17 gene, cDNA, or a subsequence thereof, e.g., the C-terminal domain.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a PRC17 polypeptide from specific species such as rat, mouse, or human can be selected to obtain only those polyclonal antibodies that are specifically immunoreactive with the PRC17 protein and not with other proteins, except for polymorphic variants and alleles of the PRC17 protein. This selection may be achieved by subtracting out antibodies that cross-react with PRC17 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind” to a protein, as defined above.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

III. Detection of PRC17 Nucleic Acids

In numerous embodiments of the present invention, nucleic acids encoding a PRC17 polypeptide, including a full-length PRC17 protein, or any derivative, variant, homolog, or fragment thereof, will be used. Such nucleic acids are useful for any of a number of applications, including for the production of PRC17 protein, for diagnostic assays, for therapeutic applications, for PRC17-specific probes, for assays for PRC17 binding and/or modulating compounds, to identify and/or isolate PRC17 homologs from other species or from mice, and other applications.

A. General Recombinant DNA Methods

Numerous applications of the present invention involve the cloning, synthesis, maintenance, mutagenesis, and other manipulations of nucleic acid sequences that can be performed using routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Isolating and Detecting PRC17 Nucleotide Sequences

In numerous embodiments of the present invention, PRC17 nucleic acids will be isolated and cloned using recombinant methods. Such embodiments are used, e.g., to isolate PRC17 polynucleotides for protein expression or during the generation of variants, derivatives, expression cassettes, or other sequences derived from PRC17, to monitor PRC17 gene expression, for the determination of PRC17 sequences in various species, for diagnostic purposes in a patient, i.e., to detect mutations in PRC17, or for genotyping and/or forensic applications.

Polymorphic variants, alleles, and interspecies homologs that are substantially identical to a PRC17 gene can be isolated using PRC17 nucleic acid probes, and oligonucleotides by screening libraries under stringent hybridization conditions. Alternatively, expression libraries can be used to clone PRC17 polymorphic variants, alleles, and interspecies homologs, by detecting expressed homologs immunologically with antisera or purified antibodies made against a PRC17 polypeptide, which also recognize and selectively bind to the PRC17 homolog.

More distantly related PRC17 homologs can be identified using any of a number of well known techniques, including by hybridizing a PRC17 probe with a genomic or cDNA library using moderately stringent conditions, or under low stringency conditions using probes from regions which are selective for PRC17, e.g., specific probes generated to the C-terminal domain. Also, a distant homolog can be amplified from a nucleic acid library using degenerate primer sets, i.e., primers that incorporate all possible codons encoding a given amino acid sequence, in particular based on a highly conserved amino acid stretch. Such primers are well known to those of skill, and numerous programs are available, e.g., on the internet, for degenerate primer design.

In certain embodiments, PRC17 polynucleotides will be detected using hybridization-based methods to determine, e.g., PRC17 RNA levels or to detect particular DNA sequences, e.g., for diagnostic purposes. For example, gene expression of PRC17 can be analyzed by techniques known in the art, e.g., Northern blotting, reverse transcription and PCR amplification of mRNA, including quantitative PCR analysis of mRNA levels with real-time PCR procedures (e.g., reverse transcriptase-TAQMAN™ amplification), dot blotting, in situ hybridization, RNase protection, probing DNA microchip arrays, and the like. In one embodiment, high density oligonucleotide analysis technology (e.g., GeneChip™) is used to identify homologs and polymorphic variants of PRC17, or to monitor levels of PRC17 mRNA. In the case where a homologs is linked to a known disease, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

Detection of PRC17 polynucloetides and polypeptides can involve quantitative or qualitative detection of the polypeptide or polynucleotide, and can involve an actual comparison with a control value or, alternatively, may be performed so that the detection itself inherently indicates an increased level of PRC17.

In certain embodiments, for example, diagnosis of cancer, the level of PRC17 polynucleotide, polypeptide, or protein activity will be quantified. In such embodiments, the difference between an elevated level of PRC17 and a normal, control level will preferably be statistically significant. Typically, a diagnostic presence, i.e., overexpression or an increase of PRC17 polypeptide or nucleic acid, represents at least about a 1.5, 2, 3, 5, 10, or greater fold increase in the level of PRC17 polypeptide or polynucleotide in the biological sample compared to a level expected in a noncancerous sample. Detection of PRC17 can be performed in vitro, i.e., in cells within a biological sample taken from the mammal, or in vivo. In one embodiment an increased level of PRC17 is used as a diagnostic marker of PRC17. As used herein, a “diagnostic presence” indicates any level of PRC17 that is greater than that expected in a noncancerous sample. In a one embodiment, assays for a PRC17 polypeptide or polynucleotide in a biological sample are conducted under conditions wherein a normal level of PRC17 polypeptide or polynucleotide, i.e., a level typical of a noncancerous sample, i.e., cancer-free, would not be detected. In such assays, therefore, the detection of any PRC17 polypeptide or nucleic acid in the biological sample indicates a diagnostic presence, or increased level.

As described below, any of a number of methods to detect PRC17 can be used. A PRC17 polynucleotide level can be detected by detecting any PRC17 DNA or RNA, including PRC17 genomic DNA, mRNA, and cDNA. A PRC17 polypeptide can be detected by detecting a PRC17 polypeptide itself, or by detecting PRC17 protein activity. Detection can involve quantification of the level of PRC17 (e.g., genomic DNA, cDNA, mRNA, or protein level, or protein activity) or, alternatively, can be a qualitative assessment of the level, or of the presence or absence, of PRC17, in particular in comparison with a control level. Any of a number of methods to detect any of the above can be used, as described infra. Such methods include, for example, hybridization, amplification, and other assays.

In certain embodiments, the ability to detect an increased level, or diagnostic presence, in a cell is used as a marker for cancer cells, i.e., to monitor the number or localization of cancer cells in a patient, as detected in vivo or in vitro.

Typically, the PRC17 polynucleotides or polypeptides detected herein will be at least about 70% identical, and preferably 75%, 80%, 85%, 86%, 87%, 88%, 89%, 90%, 91%, 92%, 93%, 94%, 95%, 96%, 97%, 98%, 99% or more identical, over a region of at least about 50, 100, 200, or more nucleotides, or 20, 50, 100, or more amino acids, to SEQ ID NO:2, SEQ ID NO:4 or SEQ ID NO:6. Such polynucleotides or polypeptides can represent functional or nonfunctional forms of PRC17, or any variant, derivative, or fragment thereof.

Detection of Copy Number

In one embodiment, e.g., for the diagnosis of cancer, the copy number, i.e., the number of PRC17 genes in a cell, is evaluated. Generally, for a given autosomal gene, an animal has two copies of each gene. The copy number can be increased, however, by gene amplification or duplication, e.g., in cancer cells, or reduced by deletion. Methods of evaluating the copy number of a particular gene are well known to those of skill in the art, and include, inter alia, hybridization- and amplification-based assays.

Hybridization-based Assays

Any of a number of hybridization-based assays can be used to detect the PRC17 gene or the copy number of PRC17 genes in the cells of a biological sample. One such method is by Southern blot. In a Southern blot, genomic DNA is typically fragmented, separated electrophoretically, transferred to a membrane, and subsequently hybridized to a PRC17-specific probe. For copy number determination, comparison of the intensity of the hybridization signal from the probe for the target region with a signal from a control probe for a region of normal genomic DNA (e.g., a nonamplified portion of the same or related cell, tissue, organ, etc.) provides an estimate of the relative PRC17 copy number. Southern blot methodology is well known in the art and is described, e.g., in Ausubel et al., or Sambrook et al., supra.

An alternative means for determining the copy number of PRC17 genes in a sample is by in situ hybridization, e.g., fluorescence in situ hybridization, or FISH. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments.

The probes used in such applications are typically labeled, e.g., with radioisotopes or fluorescent reporters. Preferred probes are sufficiently long, e.g., from about 50, 100, or 200 nucleotides to about 1000 or more nucleotides, so as to specifically hybridize with the target nucleic acid(s) under stringent conditions.

In numerous embodiments “comparative probe” methods, such as comparative genomic hybridization (CGH), are used to detect PRC17 gene amplification. In comparative genomic hybridization methods, a “test” collection of nucleic acids is labeled with a first label, while a second collection (e.g., from a healthy cell or tissue) is labeled with a second label. The ratio of hybridization of the nucleic acids is determined by the ratio of the first and second labels binding to each fiber in an array. Differences in the ratio of the signals from the two labels, e.g., due to gene amplification in the test collection, is detected and the ratio provides a measure of the PRC17 gene copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc.

Amplification-based Assays

In another embodiment, amplification-based assays are used to detect PRC17 or to measure the copy number of PRC17 genes. In such assays, the PRC17 nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the copy number of the PRC17 gene. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). The nucleic acid sequence for PRC17 (see, e.g., SEQ ID NO:1) is sufficient to enable one of skill to routinely select primers to amplify any portion of the gene.

In some embodiments, a TaqMan based assay is used to quantify PRC17 polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, for example, literature provided by Perkin-Elmer, e.g., www.perkin-elmer.com).

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Detection of PRC17 expression

Direct hybridization-based assays

Methods of detecting and/or quantifying the level of PRC17 gene transcripts (mRNA or cDNA made therefrom) using nucleic acid hybridization techniques are known to those of skill in the art (see, Sambrook et al., (1989) Molecular Cloning: A Laboratory Manual, 2d Ed., vols 1-3, Cold Spring Harbor Press, New York).

For example, one method for evaluating the presence, absence, or quantity of PRC17 cDNA involves a Northern blot. In brief, in a typical embodiment, mRNA is isolated from a given biological sample, electrophoresed to separate the mRNA species, and transferred from the gel to a nitrocellulose membrane. Labeled PRC17 probes are then hybridized to the membrane to identify and/or quantify the mRNA.

Amplification-based assays

In another embodiment, a PRC17 transcript (e.g., PRC17 mRNA) is detected using amplification-based methods (e.g., RT-PCR). RT-PCR methods are well known to those of skill (see, e.g., Ausubel et al., supra). Preferably, quantitative RT-PCR is used, thereby allowing the comparison of the level of mRNA in a sample with a control sample or value.

Detection of PRC17 Polypeptide Expression

In addition to the detection of PRC17 genes and gene expression using nucleic acid hybridization technology, PRC17 levels can also be detected and/or quantified by detecting or quantifying the polypeptide. PRC17 polypeptides are detected and quantified by any of a number of means well known to those of skill in the art. These include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. PRC17 polypeptide detection is discussed in Section V, infra.

Expression in Prokaryotes and Eukaryotes

In some embodiments, it is desirable to produce PRC17 polypeptides using recombinant technology. To obtain high level expression of a cloned gene or nucleic acid, such as a cDNA encoding a PRC17 polypeptide, a PRC17 sequence is typically subcloned into an expression vector that contains a strong promoter to direct transcription, a transcription/translation terminator, and if for a nucleic acid encoding a protein, a ribosome binding site for translational initiation. Suitable bacterial promoters are well known in the art and are described, e.g., in Sambrook et al. and Ausubel et al. Bacterial expression systems for expressing the PRC17 protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

For therapeutic applications, PRC17 nucleic acids are introduced into a cell, in vitro, in vivo, or ex vivo, using any of a large number of methods including, but not limited to, infection with viral vectors, liposome-based methods, biolistic particle acceleration (the gene gun), and naked DNA injection. Such therapeutically useful nucleic acids include, but are not limited to, coding sequences for full-length PRC17, coding sequences for a PRC17 fragment, domain, derivative, or variant, PRC17 antisense sequences, and PRC17 ribozymes. Typically, such sequences will be operably linked to a promoter, but in numerous applications a nucleic acid will be administered to a cell that is itself directly therapeutically effective, e.g., certain antisense or ribozyme molecules.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the PRC17-encoding nucleic acid in host cells. A typical expression cassette thus contains a promoter operably linked to the nucleic acid sequence encoding a PRC17 polypeptide, and signals required for efficient polyadenylation of the transcript, ribosome binding sites, and translation termination. The nucleic acid sequence encoding a PRC17 polypeptide may be linked to a cleavable signal peptide sequence to promote secretion of the encoded protein by the transfected cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which are well known to those of skill in the art.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification, such as neomycin, thymidine kinase, hygromycin B phosphotransferase, and dihydrofolate reductase. Alternatively, high yield expression systems not involving gene amplification are also suitable, such as using a baculovirus vector in insect cells, with a sequence encoding a PRC17 polypeptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of a PRC17 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of reagents such as Superfect (Qiagen), liposomes, calcium phosphate transfection, polybrene, protoplast fusion, electroporation, microinjection, plasmid vectors, viral vectors, biolistic particle acceleration (the gene gun), or any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing a PRC17 gene.

After the expression vector is introduced into the cells, the transfected cells are cultured under conditions favoring expression of the PRC17 polypeptide, which is recovered from the culture using standard techniques identified below. Methods of culturing prokaryotic or eukaryotic cells are well known and are taught, e.g., in Ausubel et al., Sambrook et al., and in Freshney, Culture of Animal Cells, 3d. Ed., (1993), A Wiley-Liss Publication.

PRC17 Transgenic Animals

Transgenic animals, including knockout transgenic animals, that include additional copies of PRC17 and/or altered or mutated PRC17 transgenes can also be generated. A “transgenic animal” refers to any animal (e.g., mouse, rat, pig, bird, or an amphibian), preferably a nonhuman mammal, in which one or more cells contain heterologous nucleic acid introduced using transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In some embodiments, PRC17 transgenic animals express a PRC17 gene, which may have an altered function with respect to a normal PRC17 gene.

In other embodiments, transgenic animals are produced in which PRC17 expression is silenced. Gene knockout by homologous recombination is a method that is commonly used to generate transgenic animals. This method requires a relatively long genomic clone of the gene to be knocked out (about 10 kb). Typically, a selectable marker is inserted into an exon of the gene of interest to effect the gene disruption, and a second counter-selectable marker provided outside of the region of homology to select homologous versus nonhomologous recombinants. This construct is transfected into embryonic stem cells and recombinants selected in culture. Recombinant stem cells are combined with very early stage embryos generating chimeric animals. If the chimerism extends to the germline homozygous knockout animals can be isolated by back-crossing.

Other methods of generating knockout animals are typified by an approach using the cre recombinase and lox DNA recognition elements. The recognition elements are inserted into a gene of interest using homologous recombination (as described above) and the expression of the recombinase induced in adult mice post-development. This causes the deletion of a portion of the target gene and typically avoids developmental complications. Transgenic mice can be derived using methodology known to those of skill in the art, see, e.g., Hogan et al., Manipulating the Mouse Embryo: A Laboratory Manual, (1988); Teratocarcinomas and Embryonic Stem Cells: A Practical Approach, Robertson, ed., (1987); and Capecchi et al., Science 244:1288 (1989).

IV. Purification of PRC17 Polypeptides

Either naturally occurring or recombinant PRC17 polypeptides can be purified for use in functional assays, binding assays, diagnostic assays, and other applications. Naturally occurring PRC17 polypeptides are purified, e.g., from mammalian tissue such as blood, lymphatic tissue, or any other source of a PRC17 homolog. Recombinant PRC17 polypeptides are purified from any suitable bacterial or eukaryotic expression system, e.g., CHO cells or insect cells.

PRC17 proteins may be purified to substantial purity by standard techniques, including, but not limited to selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant PRC17 polypeptide is being purified. For example, proteins having established molecular adhesion properties can be reversibly fused to the PRC17 polypeptide. With the appropriate ligand, a PRC17 polypeptide can be selectively adsorbed to a purification column and then freed from the column in a relatively pure form. The fused protein is then removed by enzymatic activity. PRC17 proteins can also be purified using immunoaffinity columns.

A. Purification of Recombinant PRC17 Protein

Recombinant proteins are expressed by transformed bacteria or eukaryotic cells such as CHO cells or insect cells in large amounts, typically after promoter induction but expression can be constitutive. Promoter induction with IPTG is one example of an inducible promoter system. Cells are grown according to standard procedures in the art. Fresh or frozen cells are used for isolation of protein.

Proteins expressed in bacteria may form insoluble aggregates (“inclusion bodies”). Several protocols are suitable for purification of PRC17 inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al, supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. PRC17 polypeptides are separated from other bacterial proteins by standard separation techniques, e.g., with Ni-NTA agarose resin.

Alternatively, it is possible to purify PRC17 polypeptides from bacteria periplasm. After lysis of the bacteria, when a PRC17 protein is exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying PRC17 Polypeptides

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of a PRC17 protein can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

PRC17 proteins can also be separated from other proteins on the basis of their size, net surface charge, hydrophobicity, and affinity for heterologous molecules. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

V. Antibodies to PRC17 Family Members

In numerous embodiments of the present invention, antibodies that specifically bind to PRC17 polypeptides will be used. Such antibodies have numerous applications, including for the modulation of PRC17 activity and for immunoassays to detect PRC17, and variants, derivatives, fragments, etc. of PRC17. Immunoassays can be used to qualitatively or quantitatively analyze the PRC17 polypeptide. A general overview of the applicable technology can be found in Harlow & Lane, Antibodies: A Laboratory Manual (1988).

Methods of producing polyclonal and monoclonal antibodies that react specifically with PRC17 polypeptides are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of PRC17-comprising immunogens can be used to produce antibodies specifically reactive with a PRC17 polypeptide. For example, a recombinant PRC17 protein, or an antigenic fragment thereof, is isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein can also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the PRC17 polypeptide. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-PRC17 proteins, or even related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

A. Immunological Binding Assays

Once PRC17-specific antibodies are available, individual PRC17 proteins can be detected by a variety of immunoassay methods. For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Moreover, the immunoassays of the present invention can be performed in any of several configurations, which are reviewed extensively in Enzyme Immunoassay (Maggio, ed., 1980); and Harlow & Lane, supra. Immunological binding assays (or immunoassays) typically use an antibody that specifically binds to a protein or antigen of choice (in this case a PRC17 protein or an antigenic subsequence thereof). The antibody (e.g., anti-PRC17) may be produced by any of a number of means well known to those of skill in the art and as described above.

Immunoassays also often use a labeling agent to specifically bind to and label the complex formed by the antibody and antigen. The labeling agent can itself be one of the moieties comprising the antibody/antigen complex. Thus, the labeling agent can be a labeled PRC17 polypeptide or a labeled anti-PRC17 antibody. Alternatively, the labeling agent can be a third moiety, such a secondary antibody, that specifically binds to the antibody/PRC17 complex (a secondary antibody is typically specific to antibodies of the species from which the first antibody is derived). Other proteins capable of specifically binding immunoglobulin constant regions, such as protein A or protein G, can also be used as the label agent. These proteins exhibit a strong nonimmunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

1. Noncompetitive assay formats

Immunoassays for detecting a PRC17 protein in a sample can be either competitive or noncompetitive. Noncompetitive immunoassays are assays in which the amount of antigen is directly measured. In one preferred “sandwich” assay, for example, the anti-PRC17 antibodies can be bound directly to a solid substrate on which they are immobilized. These immobilized antibodies then capture the PRC17 protein present in the test sample. The PRC17 protein thus immobilized is then bound by a labeling agent, such as a second PRC17 antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

2. Competitive assay formats

In competitive assays, the amount of PRC17 protein present in the sample is measured indirectly by measuring the amount of a known, added (exogenous) PRC17 protein displaced (competed away) from an anti-PRC17 antibody by the unknown PRC17 protein present in a sample. In one competitive assay, a known amount of PRC17 protein is added to a sample and the sample is then contacted with an antibody that specifically binds to the PRC17 protein. The amount of exogenous PRC17 protein bound to the antibody is inversely proportional to the concentration of PRC17 protein present in the sample. In a particularly preferred embodiment, the antibody is immobilized on a solid substrate. The amount of PRC17 protein bound to the antibody may be determined either by measuring the amount of PRC17 protein present in a PRC17/antibody complex, or alternatively by measuring the amount of remaining uncomplexed protein. The amount of PRC17 protein may be detected by providing a labeled PRC17 molecule.

A hapten inhibition assay is another preferred competitive assay. In this assay, the known PRC17 protein is immobilized on a solid substrate. A known amount of anti-PRC17 antibody is added to the sample, and the sample is then contacted with the immobilized PRC17. The amount of anti-PRC17 antibody bound to the known immobilized PRC17 protein is inversely proportional to the amount of PRC17 protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

3. Cross-reactivity determinations

Immunoassays in the competitive binding format can also be used for crossreactivity determinations. For example, a protein at least partially encoded by SEQ ID NO:2 can be immobilized to a solid support. Proteins (e.g., PRC17 proteins and homologs) are added to the assay that compete for binding of the antisera to the immobilized antigen. The ability of the added proteins to compete for binding of the antisera to the immobilized protein is compared to the ability of the PRC17 polypeptide encoded by SEQ ID NO:2 to compete with itself. The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of a PRC17 protein, to the immunogen protein (i.e., PRC17 protein encoded by SEQ ID NO:2). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by SEQ ID NO:2 that is required to inhibit 50% of binding, then the second protein is said to specifically bind to the polyclonal antibodies generated to a PRC17 immunogen.

Polyclonal antibodies that specifically bind to a PRC17 protein from a particular species can be make by subtracting out cross-reactive antibodies using PRC17 homologs. For example, antibodies specific to human PRC17 can be made by subtracting out antibodies that are cross-reactive with mouse PRC17. In an analogous fashion, antibodies specific to a particular PRC17 protein can be made in an organism with multiple PRC17 genes.

4. Other assay formats

Western blot (immunoblot) analysis is used to detect and quantify the presence of PRC17 protein in a sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the PRC17 protein. The anti-PRC17 polypeptide antibodies specifically bind to the PRC17 polypeptide on the solid support. These antibodies may be directly labeled or alternatively may be subsequently detected using labeled antibodies (e.g., labeled sheep anti-mouse antibodies) that specifically bind to the anti-PRC17 antibodies.

Additional assay include immunocytochemical assays that identify the presence of PRC17 in particular cells and the subcellular localization of PRC17. Such assay are performed using standard techniques (see, e.g., Current Protocols in Immunology, 1991) Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

5. Reduction of nonspecific binding

One of skill in the art will appreciate that it is often desirable to minimize nonspecific binding in immunoassays. Particularly, where the assay involves an antigen or antibody immobilized on a solid substrate it is desirable to minimize the amount of nonspecific binding to the substrate. Means of reducing such nonspecific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

6. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵i, ³⁵s, ¹⁴C, or ³²p), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Nonradioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize a PRC17 protein, or secondary antibodies that recognize anti-PRC17.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluninescent compounds include luciferin, and 2,3-dihydrophthalazinediones, e.g., luminol. For a review of various labeling or signal producing systems that may be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

VI. Diagnosis of Diseases Associated with Altered PRC17 Activity or Expression

PRC17 nucleic acids, proteins, and/or antibodies can be used diagnostically or prognostically to detect diseases or conditions associated with altered PRC17 activity or expression relative to normal, for example cancer. Such diseases can be associated with either decreased or increased PRC17 activity or expression. PRC17 activity or expression can be detected using any of a variety of reagents including, for example, PRC17 protein, mRNA, genomic DNA, or antibodies to PRC17. Changes in activity can indicate alterations in, e.g., PRC17 gene copy number, transcription, translation, RNA or protein stability, or protein activity. Accordingly, any of a large number of assays can be used to detect the PRC17 nucleic acids or polypeptides.

In one embodiment, for example, a PRC17 nucleic acid or protein can be used as a diagnostic or prognostic tool, alone or in combination with other diagnostic methods, to detect increases in PRC17 copy number or expression that are associated with cancer, e.g., prostate, colon, ovarian or breast as well as other cancers such as lung, kidney, stomach, bladder, or a cancer of the gastrointestinal tract. The detection of PRC17 nucleic acids or proteins can also be used to monitor the efficacy of a cancer treatment. For example, the level of PRC17 protein or nucleic acid after an anti-cancer treatment can be compared to the level before treatment, wherein a decrease in the level of the PRC17 protein or nucleic acid after the treatment indicates efficacious treatment. The levels of PRC17 protein or nucleic acid can also be used to influence the choice of anti-cancer treatment in a mammal, where, for example, a large increase in PRC17 indicates the use of a more aggressive anti-cancer therapy, and a small increase or no increase indicates the use of a less aggressive anti-cancer therapy. In addition, the ability to detect cancer cells that exhibit altered PRC17 activity or expression can be useful in monitoring, e.g., in vivo or in vitro, the number and/or location of cancer cells in a patient in order to assess the progression of the disease over time.

VII. Modulating PRC17 Activity

A. Assays for Modulators of PRC17 Proteins

In numerous embodiments of this invention, the level of PRC17 activity will be modulated in a cell by administering to the cell, in vivo or in vitro, any of a large number of PRC17-modulating molecules, e.g., polypeptides, antibodies, amino acids, nucleotides, lipids, carbohydrates, or any organic or inorganic molecule.

To identify molecules capable of modulating PRC17, assays will be performed to detect the effect of various compounds on PRC17 activity in a cell. The activity of PRC17 polypeptides can be assessed using a variety of in vitro and in vivo assays to determine functional, chemical, and physical effects, e.g., measuring the binding of PRC17 to other molecules (e.g., radioactive binding), measuring PRC17 protein and/or RNA levels, or measuring other aspects of PRC17 polypeptides, e.g., phosphorylation levels, transcription levels, receptor activity, ligand binding and the like. Such assays can be used to test for both activators and inhibitors of PRC17 proteins. Modulators thus identified are useful for, e.g., many diagnostic and therapeutic applications.

The PRC17 protein of the assay will typically be a recombinant or naturally occurring polypeptide with a sequence of SEQ ID NO:2, SEQ ID NO:4 or SEQ ID NO:6 or a conservatively modified variant thereof. Alternatively, the PRC17 protein of the assay will be derived from a eukaryote and include an amino acid subsequence having amino acid sequence identity to SEQ ID NO:2, SEQ ID NO:4 or SEQ ID NO:6. Generally, the amino acid sequence identity will be at least 70%, optionally at least 75%, 85%, or 86%, 87%, 88%, 89%, 90 %, 91%, 92%, 93%, 94%, 95%, 96%, 97%, 98% or greater. Optionally, the polypeptide of the assays will comprise a domain of a PRC17 protein, such as an extracellular loop,or one or more transmembrane domains and the like. In certain embodiments, a domain of a PRC17 protein, e.g., a transmembrane domain or extracellular loop, is bound to a solid substrate and used, e.g., to isolate any molecules that can bind to and/or modulate their activity. In certain embodiments, a domain of a PRC17 polypeptide, e.g., an N-terminal domain, a C-terminal domain, an extracellular loop, or one or more transmembrane domains, is fused to a heterologous polypeptide, thereby forming a chimeric polypeptide. Such chimeric polypeptides are also useful, e.g., in assays to identify modulators of PRC17.

Samples or assays that are treated with a potential PRC17 protein inhibitor or activator are compared to control samples without the test compound, to examine the extent of modulation. Control samples (untreated with activators or inhibitors) are assigned a relative PRC17 activity value of 100. Inhibition of a PRC17 protein is achieved when the PRC17 activity value relative to the control is about 90%, optionally about 50%, optionally about 25-0%. Activation of a PRC17 protein is achieved when the PRC17 activity value relative to the control is about 110%, optionally about 150%, 200-500%, or about 1000-2000%.

The effects of the test compounds upon the function of the polypeptides can be measured by examining any of the parameters described above. Any suitable physiological change that affects PRC17 activity can be used to assess the influence of a test compound on the polypeptides of this invention. When the functional consequences are determined using intact cells or animals, one can also measure a variety of effects such as changes in cell growth or changes in cell-cell interactions.

Modulators of PRC17 that act by modulating PRC17 gene expression can also be identified. For example, a host cell containing a PRC17 protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured using any method known to those of skill in the art to be suitable. For example, mRNA expression of the protein of interest may be detected using Northern blots or by detecting their polypeptide products using immunoassays.

B. Assays for PRC17-Interacting Compounds

In certain embodiments, assays will be performed to identify molecules that physically interact with PRC17 proteins. Such molecules can be any type of molecule, including polypeptides, polynucleotides, amino acids, nucleotides, carbohydrates, lipids, or any other organic or inorganic molecule. Such molecules may represent molecules that normally interact with PRC17 or may be synthetic or other molecules that are capable of interacting with PRC17 and that can potentially be used as lead compounds to identify classes of molecules that can interact with and/or modulate PRC17. Such assays may represent physical binding assays, such as affinity chromatography, immunoprecipitation, two-hybrid screens, or other binding assays, or may represent genetic assays.

In any of the binding or functional assays described herein, in vivo or in vitro, any PRC17 protein, or any derivative, variation, homolog, or fragment of a PRC17 protein, can be used. Preferably, the PRC17 protein has at least about 85% identity to an amino acid sequence of SEQ ID NO:2 or at least 70% identity to an amino acid sequence of SEQ ID NO:4 or SEQ ID NO:6. In numerous embodiments, a fragment of a PRC17 protein is used. For example, a fragment that contains only an N-terminal or C-terminal domain, or an extracellular loop or transmembrane domain can be used. Such fragments can be used alone, in combination with other PRC17 fragments, or in combination with sequences from heterologous proteins, e.g., the fragments can be fused to a heterologous polypeptides, thereby forming a chimeric polypeptide.

Compounds that interact with PRC17 proteins can be isolated based on an ability to specifically bind to a PRC17 protein or fragment thereof. In numerous embodiments, the PRC17 protein or protein fragment will be attached to a solid support. In one embodiment, affinity columns are made using the PRC17 polypeptide, and physically-interacting molecules are identified. It will be apparent to one of skill that chromatographic techniques can be performed at any scale and using equipment from many different manufactures (e.g., Pharmacia Biotechnology). In addition, molecules that interact with PRC17 proteins in vivo can be identified by co-immunoprecipitation or other methods, i.e., immunoprecipitating PRC17 protein using anti-PRC17 antibodies from a cell or cell extract, and identifying compounds, e.g., proteins, that are precipitated along with the PRC17 protein. Such methods are well known to those of skill in the art and are taught, e.g., in Ausubel et al., Sambrook et al., and Harlow & Lane, all supra.

C. Modulators and Binding Compounds

The compounds tested as modulators of a PRC17 protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

1. Solid state and soluble high throughput assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, PRC17 protein, or cell or tissue expressing a PRC17 protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Nonchemical approaches for fixing tag binders to substrates include other common methods, such as heat, cross-linking by UV radiation, and the like.

2. Computer-based assays

Yet another assay for compounds that modulate PRC17 protein activity involves computer assisted drug design, in which a computer system is used to generate a three-dimensional structure of a PRC17 protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a PRC17 polypeptide into the computer system. The nucleotide sequence encoding the polypeptide, or the amino acid sequence thereof, is preferably SEQ ID NO:2, and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the PRC17 protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of PRC17 genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated PRC17 genes involves receiving input of a first nucleic acid or amino acid sequence of SEQ ID NO:1 or SEQ ID NO:2, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various PRC17 genes, and mutations associated with disease states and genetic traits.

VIII. Modulating PRC17 Activity/Expression to Treat Diseases or Conditions

In numerous embodiments of this invention, a compound, e.g., nucleic acid, polypeptide, or other molecule is administered to a patient, in vivo or ex vivo, to effect a change in PRC17 activity or expression in the patient. The desired change may be either an increase or a decrease in activity or expression of PRC17. For example, in a cancer patient with a tumor that exhibits increased levels of PRC17 relative to normal tissue, it may be desirable to decrease the activity or expression of PRC17. In other embodiments of the invention, antibodies that block PRC17 activity or function can be administered to a patient with a PRC17-expressing tumor to inhibit PRC17 function at the cell membrane surface and thus inhibit tumor growth, migration, or metastasis. In other patients with diseases associated with decreased activity or expression of PRC17, it may be desirable to increase the activity or expression of PRC17.

Compounds that can be administered to a patient include nucleic acids encoding full length PRC17 polypeptides, e.g., as shown as SEQ ID NO:1, or any derivative, fragment, or variant thereof, operably linked to a promoter. Suitable nucleic acids also include inhibitory sequences such as antisense or ribozyme sequences, which can be delivered in, e.g., an expression vector operably linked to a promoter, or can be delivered directly. Also, any nucleic acid that encodes a polypeptide that modulates the expression of PRC17 can be used. In general, nucleic acids can be delivered to cells using any of a large number of vectors or methods, e.g., retroviral, adenoviral, or adeno-associated virus vectors, liposomal formulations, naked DNA injection, and others. All of these methods are well known to those of skill in the art.

Proteins can also be delivered to a patient to modulate PRC17 activity. In preferred embodiments, a polyclonal or monoclonal antibody that specifically binds to PRC17 will be delivered. In addition, any polypeptide that interacts with and/or modulates PRC17 activity can be used, e.g., a polypeptide that is identified using the presently described assays. In addition, polypeptides that affect PRC17 expression can be used.

Further, any compound that is found to or designed to interact with and/or modulate the activity of PRC17 can be used. For example, any compound that is found, using the methods described herein, to bind to or modulate the activity of PRC17 can be used.

Any of the above-described molecules can be used to increase or decrease the expression or activity of PRC17, or to otherwise affect the properties and/or behavior of PRC17 polypeptides or polynucleotides, e.g., stability, intracellular localization, interactions with other intracellular or extracellular moieties, etc.

A. Administration and Pharmaceutical Compositions

Administration of any of the present molecules can be achieved by any of the routes normally used for introducing or bringing a modulator compound into ultimate contact with the tissue to be treated. The modulators are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The PRC17 modulators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and nonaqueous solutions, isotonic sterile solutions, which can contain antioxidants, buffers, bacteriostats, and solutes that render the formulation isotonic, and aqueous and nonaqueous sterile suspensions that can include suspending agents, solubilizers, thickening agents, stabilizers, and preservatives. In the practice of this invention, compositions can be administered, for example, orally, nasally, topically, intravenously, intraperitoneally, intravesically or intrathecally. The formulations of compounds can be presented in unit-dose or multi-dose sealed containers, such as ampules and vials. Solutions and suspensions can be prepared from sterile powders, granules, and tablets of the kind previously described. The modulators can also be administered as part a of prepared food or drug.

The dose administered to a patient, in the context of the present invention should be sufficient to effect a beneficial response in the subject over time. The dose will be determined by the efficacy of the particular modulators employed and the condition of the subject, as well as the body weight or surface area of the area to be treated. The size of the dose also will be determined by the existence, nature, and extent of any adverse side-effects that accompany the administration of a particular compound or vector in a particular subject.

In determining the effective amount of the modulator to be administered, a physician may evaluate circulating plasma levels of the modulator, modulator toxicities, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the compound at various concentrations, as applied to the mass and overall health of the subject. Administration can be accomplished via single or divided doses.

IX. Kits

Reagents that specifically hybridize to PRC17 nucleic acids, such as PRC17 probes and primers, and PRC17-specific reagents that specifically bind to or modulate the activity of a PRC17 protein, e.g., PRC17 antibodies or other compounds are used to treat PRC17-associated diseases or conditions.

Nucleic acid assays for detecting the presence of DNA and RNA for a PRC17 polynucleotide in a sample include numerous techniques known to those skilled in the art, such as Southern analysis, Northern analysis, dot blots, RNase protection, S1 analysis, amplification techniques such as PCR and LCR, and in situ hybridization. In in situ hybridization, for example, the target nucleic acid is liberated from its cellular surroundings so as to be available for hybridization within the cell while preserving the cellular morphology for subsequent interpretation and analysis. The following articles provide an overview of the art of in situ hybridization: Singer et al., Biotechniques 4:230-250 (1986); Haase et al., Methods in Virology, vol. VII, pp. 189-226 (1984); and Nucleic Acid Hybridization: A Practical Approach (Hames et al., eds. 1987). In addition, a PRC17 protein can be detected using the various immunoassay techniques described above. The test sample is typically compared to both a positive control (e.g., a sample expressing a recombinant PRC17 protein) and a negative control.

The present invention also provides for kits for screening for modulators of PRC17 proteins or nucleic acids. Such kits can be prepared from readily available materials and reagents. For example, such kits can comprise any one or more of the following materials: PRC17 nucleic acids or proteins, reaction tubes, and instructions for testing PRC17 activity. Optionally, the kit contains a biologically active PRC17 protein. A wide variety of kits and components can be prepared according to the present invention, depending upon the intended user of the kit and the particular needs of the user.

### EXAMPLES

The following examples are provided by way of illustration only and not by way of limitation. Those of skill in the art will readily recognize a variety of noncritical parameters that could be changed or modified to yield essentially similar results.

### Example 1

### PRC17 is Amplified and Overexpressed in Cancer Relative to Normal

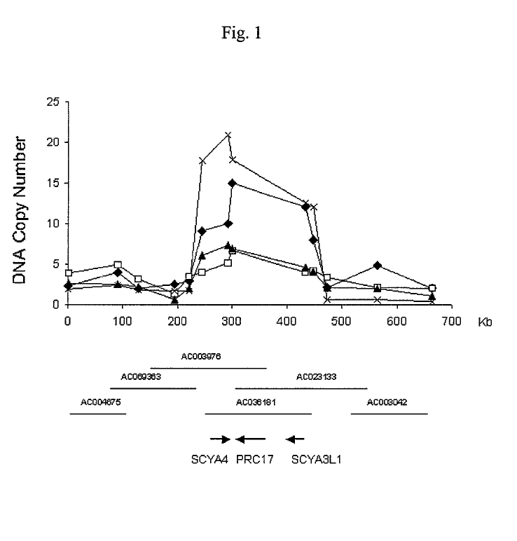

PRC17 was identified in the amplified epicenter of a particular chromosal region that is amplified in cancer (FIG. 1). Based on the Human Genone Project draft sequence data, PRC17 maps to 17q11-12. The epicenter was discovered by microarry analysis (e.g., Pollack et al., Nature Genetics 23:41-46 (1999)) and defined by quantitative PCR. PRC17 was then identified by homolog searching the epicenter genomic sequence using bioinformatics tool.

Primary tumor sample and tumor cell lines were then analyzed for amplification of PRC17 using PCR. DNA copy number was measured by quantitative PCR using a TaqMan protocol (Perkin Elmer). Briefly, DNA was purified from tumor cell lines, primary tumors or metastatic tumors. The DNA copy number of PRC17 in each sample was directly measured using PCR and a fluorescence-labeled probed. The number of PCR cycles needed to cross a preset threshold, also known as Ct value, in the sample tumor DNA preparations and a series of normal human DNA preparations at various concentrations was measured for both the target probe and a known single-copy DNA probe by using a PE/ABI 7700 TaqMan machine. The relative abundance of target sequence to the single-copy probe in each sample was then calculated by statistical analyses of the Ct values of the unknown samples and the standard curve generated from the normal human DNA preparations at various concentrations.

A fragment of PRC17 DNA was amplified using the following primer set: a forward primer of the sequence 5′-GGAGGGAACTGAGAACTTTCCA-3′ (SEQ ID NO:7), which anneals between residues 174 and 195 with a Tm of 58° C., and a reverse primer, 5′-CGAACAGCAGTATGCTCCCAC-3′ (SEQ ID NO:8), which anneals between residues 245 and 225 with a Tm of 59° C. PCR was performed using 5 nM of template DNA and 50 μM of primer DNA in 50 mM salt and 1 mM Mg⁺⁺. A three step PCR was performed for 35 cycles. Denaturation was at 94° C. for 20 second, annealing was performed at 55° C. for 20 seconds and extension was performed at 72° C. for 30 seconds. A fluorescent-labeled Taqman probe, 5′-Fam-TCAGGGCCTTGCCTCTGCGG-TAMRA-3′ (SEQ ID NO:9), which anneals between residues 204 and 223 with a Tm of 69° C., was included in the reaction for quantitation.

The RT-Taqman showed that PRC17 was overexpressed in six out of nine metastatic prostate samples analyzed (using 3-fold overexpression as cutoff) (Table 1). The highest fold overexpression is over 100 fold. The expression of PRC17 was not elevated in the amplified primary prostate tumor sample PP480. It is thought that this was due to the fact that mRNA of PRC17 was not stable in that sample.

TABLE 1
Amplification and overexpression of PRC17 in metastatic and primary
prostate tumors

| | | PRC17 | |
|---|---|---|---|
| | Tumor type | Fold of amplification | Fold of expression |
| WA4-1 | Metastatic | 2.2 | 2 |
| WA4-3 | Metastatic | 2.3 | 33.3 |
| WA5-1 | Metastatic | 1.8 | 45.7 |
| WA5-3 | Metastatic | 1.3 | 8.7 |
| WA5-4 | Metastatic | 1.5 | 13.9 |
| WA13-1 | Metastatic | 2.1 | 17.9 |
| WA12-2 | Metastatic | 2 | >100 |
| WA20-10 | Metastatic | 2.2 | 2.1 |
| WA20-45 | Metastatic | 2.1 | 1.8 |
| PP480 | Primary | 18.7 | 1.58 |
| PP486 | Primary | 6.7 | 2.137 |

In addition, expression status and amplification status of PRC17 in various breast cancer cell lines show good correlation (Table 2).

TABLE 2
Amplification and overexpression of PRC17 in breast cancer cell lines

PRC17

| | Folds of Amplification | Folds of Expression |
|---|---|---|
| AlaB | 3.1 | 2.45 |
| BT20 | 2.8 | 0.9 |
| ZR75-30 | 4.5 | 18 |
| BT474 | 6.9 | 6.3 |
| Du4475 | 3.1 | 1.2 |
| MDA MB330 | 22.5 | 66 |

PRC17 amplification was studied in additional primary and metastatic prostate tumors, and in breast, lung, and ovarian tumors. The results are summarized in Table 3.

TABLE 3
Amplification and overexpression status of PRC17 in the major tumor types

| | Primary | Metastatic | | | |
|---|---|---|---|---|---|
| | prostate | prostate | Breast | Lung | Ovary |
| Amplification¹ | 15% (3/20) | 0% (0/15) | 1% (1/99) | 0% (0/26) | 13% (4/31) |
| | (20×)³ | | (5×) | | (5×) |
| Overexpression² | 12% (2/17) | 53% (8/15) | 19% (3/16) | 18% (4/22) | 9% (1/11) |
| | (3.3×) | (>100×) | (13×) | (5×) | (11×) |

¹DNA amplification cutoff: 2.5×
²RNA overexpression cutoff: 3×
³The highest observed amplification and overexpression

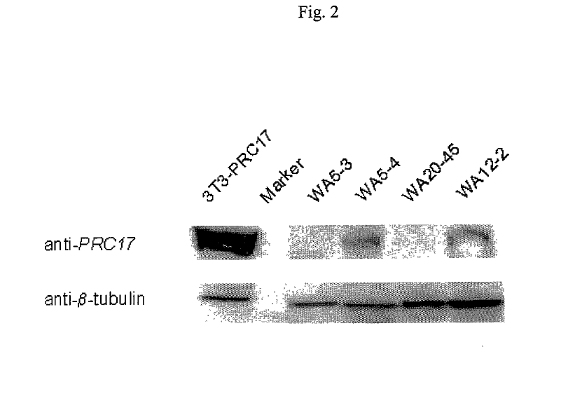

Levels of PRC17 protein were also examined in metastatic prostate tumor samples. PRC17 protein levels were determined. PRC17 protein levels in control 3T3 cells stably transfected with PRC17 and in metastatic prostate tumor samples prostate tumor samples (WA5-3, WA5-4, WA20-45, and WA12-2) were measured by Western blot using an anti-PRC17 polyclonal antibody to detect endogenous PRC17. The rabbit polyclonal antibody was directed against a unique C-terminal peptide in PRC17 (NH2-PSTSDQGTPFRARDEQPC-OH, Antibody Solution, Palo Alto, Calif.). β-tubulin antibody was used as a control for loading efficiency. The results show that two of the metastatic prostate tumors that overexpressed the gene, WA5-4 and WA12-2, also overexpressed the protein (FIG. 2).

### Example 2

### PRC17 Interacts With Rab5

The PRC17 protein sequence contains a domain that has been shown to encode the catalytic core of GTPase activating protein (GAP). This domain is also found in mammalian RN-Tre, a Rab5 GAP. To test whether PRC17 acts as a Rab5 GAP, cell lines were established that stably express either the wild type PRC17, or a mutant form PRC17 in which two conserved amino acids Arg¹⁰⁷ and Asp¹⁴⁸ were substituted by alanines. Point mutations of the corresponding amino acids on RN-Tre have been show to abolish GAP activity of that protein. This mutant form of PRC17 was used as a control in the following experiments.

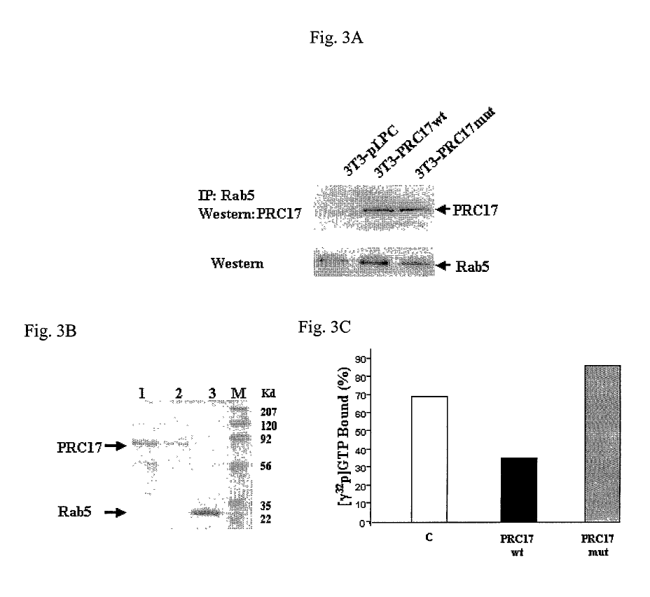

Proteins were immunoprecipitated with anti-Rab5 antibody. As shown in FIG. 3A (upper panel), PRC17 protein was detected in immunocomplexes from cells expressing either the wild type or mutant PRC17, whereas no PRC17 protein was present in the immunocomplex from cells expressing the vector. Western blot analysis showed that Rab5 was present in all cell lines (FIG. 3A, lower panel). Thus, PRC17 interacts with Rab5 and point mutations within the PRC17 Rab GAP domain have no effect on protein-protein interaction between PRC17 and Rab5.

To test whether PRC17 is a Rab5 GAP, we performed GTP-hydrolysis assays using recombinant PRC17 and Rab5 proteins (FIG. 3B). As shown in FIG. 3C, about 70% of Rab5 protein was GTP-bound after a 5-min incubation in the absence of PRC17 protein. Addition of wild type PRC17 resulted in a 30% reduction of GTP-bound Rab5 whereas addition of the mutant form PRC17 had no effect on Rab5 GTP-hydrolysis. These results demonstrate that PRC17 activates Rab5 GTPase activity and that point mutations within the Rab GAP domain abolished GAP activity of PRC17.

### Example 3

### PRC17 has Oncogenic Properties

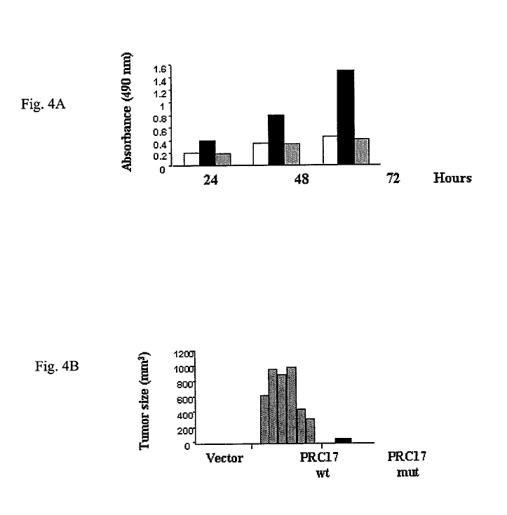

PRC17 confers growth advantage in low serum. To characterize the oncongenic properties of PRC17, the role of PRC17 in cell proliferation was determined using a non-radioactive cell proliferation assay. As shown in FIG. 4A, when cells were cultured in 0.5% serum, 3T3 cells expressing either vector alone or the mutant form of PRC17 stopped dividing after 48 hour. However, cells expressing wild type PRC17 proliferated rapidly in the low serum, resulting in a 3-fold increase in cell number after 72 hours in culture. These results indicate that PRC17 confers growth advantage in low serum and that this function requires a functional GAP domain.

PRC17 induces foci. The ability of PRC17 protein to induce transformation was analyzed by determining its ability to form foci in monolayer cell culture. 3T3 cells were transiently transfected with either vector alone or an expression vector expressing wild type PRC17, and then grown under reduced serum conditions for 2 to 3 weeks. Only the PRC17 transfected cells developed foci after 2 weeks in culture, when cells had become confluent. This result indicates that cells over-expressing PRC17 have reduced contact inhibition.

Tumorogenesis of PRC17 in nude mice. To determine whether PRC17 is tumorigenic in vivo, 3T3 cells expressing vector alone, wild type or mutant PRC17 were injected subcutaneously into athymic nude mice. As shown in FIG. 4C, all animals injected with 3T3 cells expressing wild type PRC17 developed large tumors within 4 weeks. No mouse injected with cells expressing vector alone developed tumors. Of the mice injected with cells expressing mutant PRC17, only one developed a small tumor after 4 weeks. These results indicate that PRC17 is a potent transforming gene in vivo and that GAP activity is required for its transforming function.

### Example 4

### Splice Variant 1

Similar studies were carried out to analyze overexpression for PRC17 Splice Variant 1. A fragment of PRC17 Splice Variant 1 DNA was amplified using the following primer set: a forward primer of the sequence 5′-GGATATGGCACGGACCCA-3′ (SEQ ID NO:10), which anneals between residues 362 and 379 with a Tm of 59° C., and a reverse primer, 5′-GGACCTGGACGTAGAGGGC-3′ (SEQ ID NO:11), which anneals between residues 434 and 416 with a Tm of 58° C. PCR was performed using 5 nM of template DNA and 50 μM of primer DNA in 50 mM salt and 1 mM Mg⁺⁺. A three step PCR was performed for 35 cycles. Denaturation was at 94° C. for 20 second, annealing was performed at 55° C. for 20 seconds and extension was performed at 72° C. for 30 seconds. A fluorescent-labeled Taqman probe, 5′-Fam-TCTGTCTGAAATCATAATGGCGGAACCAA-TAMRA-3′ (SEQ ID NO:12), which anneals between residues 385 and 413 with a Tm of 68° C., was included in the reaction for quantitation.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### Table of Nucleic Acid and Polypeptide Sequences

What is claimed is:

1. A method of detecting cancer cells in a biological sample from a mammal, the method comprising steps of:
(i) providing the biological sample from the mammal; and
(ii) detecting a nucleic acid molecule encoding a PRC17 polypeptide comprising at least 85% amino acid sequence identity to an amino acid sequence of SEQ ID NO:2 or at least 70% amino acid identity to an amino acid sequence of SEQ ID NO:4 or SEQ ID NO:6 in the biological sample, wherein an increase in the level of the nucleic acid molecule in the sample compared to normal indicates the presence of cancer cells.

2. The method of claim 1, wherein the polypeptide has an amino acid sequence of SEQ ID NO:2, SEQ ID NO:4 or SEQ ID NO:6.

3. The method of claim 1, wherein the detecting step further comprises:
(a) contacting the nucleic acid molecule with a probe under conditions in which the probe selectively hybridizes to the nucleic acid molecule to form a stable hybridization complex; and
(b) detecting the hybridization complex.

4. The method of claim 3, wherein the contacting step further comprises a step of amplifying the gene in an amplification reaction.

5. The method of claim 4, wherein the amplification reaction is a polymerase chain reaction.

6. The method of claim 1, wherein the nucleic acid is an mRNA.

7. The method of claim 1, wherein the biological sample is a tissue biopsy.

8. The method of claim 7, wherein the cancer cells are selected from the group consisting of prostate tissue, breast tissue, lung tissue, and ovarian tissue.

9. The method of claim 1, wherein the mammal is a human.

10. A method of detecting a presence of cancer cells in a biological sample from a mammal, the method comprising steps of:
(i) providing the biological sample from the mammal; and
(ii) detecting an overexpression of a polypeptide comprising polypeptide comprising at least 85% amino acid sequence identity to an amino acid sequence of SEQ ID NO:2 or at least 70% amino acid identity to an amino acid sequence of SEQ ID NO:4 or SEQ ID NO:6 in the biological sample, thereby detecting the presence of cancer cells in the biological sample.

11. The method of claim 10, wherein the polypeptide has an amino acid sequence of SEQ ID NO:2, SEQ ID NO:4 or SEQ ID NO:6.

12. The method of claim 10, wherein the polypeptide is detected using an antibody that selectively binds to the polypeptide.

13. The method of claim 10, wherein the biological sample is a tissue biopsy.

14. The method of claim 10, wherein the cancer cells are selected from the group consisting of prostate cancer cells, breast cancer cells, lung cancer cells, and ovarian cancer cells.

15. The method of claim 10, wherein the mammal is a human.

16. A method of monitoring the efficacy of a therapeutic treatment of a cancer, the method comprising the steps of:
(i) providing a biological sample from a mammal undergoing the therapeutic treatment; and
(ii) detecting a level of a polypeptide comprising at least 85% amino acid sequence identity to an amino acid sequence of SEQ ID NO:2 or at least 70% amino acid identity to an amino acid sequence of SEQ ID NO:4 or SEQ ID NO:6 in the biological sample compared to a level in a biological sample from the mammal prior to, or earlier in, the therapeutic treatment, thereby monitoring the efficacy of the therapy.

17. The method of claim 16, wherein the polypeptide has an amino acid sequence of SEQ ID NO:2, SEQ ID NO:4 or SEQ ID NO:6.

18. The method of claim 16, wherein the cancer is selected from the group consisting of prostate cancer, ovarian cancer, lung cancer, and breast cancer.

19. The method of claim 16, wherein the polypeptide is detected using an antibody that selectively binds to the polypeptide.

20. A method of monitoring the efficacy of a therapeutic treatment of a cancer, the method comprising the steps of:
(i) providing a biological sample from a mammal undergoing the therapeutic treatment; and
(ii) detecting a nucleic acid molecule encoding a PRC17 polypeptide comprising at least 85% amino acid sequence identity to an amino acid sequence of SEQ ID NO:2 or at least 70% amino acid identity to an amino acid sequence of SEQ ID NO:4 or SEQ ID NO:6 in the biological sample compared to a level in a biological sample from the mammal prior to, or earlier in, the therapeutic treatment, thereby monitoring the efficacy of the therapy.

21. An isolated nucleic acid encoding a PRC17 polypeptide, the nucleic acid encoding a polypeptide comprising at least 85% amino acid identity to an amino acid sequence of SEQ ID NO:2 or at least 70% identity to an amino acid sequence of SEQ ID NO:4 or SEQ ID NO:6.

22. The nucleic acid of claim 21, wherein the nucleic acid encodes a PRC17 polypeptide that specifically binds to polyclonal antibodies generated against an amino acid sequence of SEQ ID NO:2, SEQ ID NO:4 or SEQ ID NO:6.

23. The nucleic acid of claim 21, wherein the nucleic acid encodes a PRC17 polypeptide comprising an amino acid sequence of SEQ ID NO:2, SEQ ID NO:4 or SEQ ID NO:6.

24. The nucleic acid of claim 23, wherein the nucleic acid comprises a nucleotide sequence of SEQ ID NO:1, SEQ ID NO:3 or SEQ ID NO:5.

25. The nucleic acid of claim 21, wherein the nucleic acid is amplified by primers that specifically hybridize under stringent hybridization conditions to a nucleic acid having a nucleotide sequence of SEQ ID NO:1, SEQ ID NO:3 or SEQ ID NO:5.

26. The nucleic acid of claim 21, wherein the nucleic acid specifically hybridizes under stringent hybridization conditions to a nucleic acid having a nucleotide sequence of SEQ ID NO:1, SEQ ID NO:3 or SEQ ID NO:5.

27. An isolated PRC17 polypeptide, the polypeptide comprising at least 85% amino acid sequence identity to an amino acid sequence of SEQ ID NO:2 or at least 70% amino acid identity to an amino acid sequence of SEQ ID NO:4 or SEQ ID NO:6.

28. The isolated polypeptide of claim 8, wherein the polypeptide specifically binds to polyclonal antibodies generated against SEQ ID NO:2, SEQ ID NO:4 or SEQ ID NO:6.

29. The isolated polypeptide of claim 8, wherein the polypeptide has an amino acid sequence of SEQ ID NO:2, SEQ ID NO:4 or SEQ ID NO:6.

30. An antibody that selectively binds to the polypeptide of claim 8.

31. An expression vector comprising the nucleic acid of claim 1.

32. A host cell transfected with the vector of claim 31.

33. A method of identifying a compound that modulates activity of a PRC17 polypeptide, the method comprising steps of:
(i) contacting the polypeptide with the compound, wherein the polypeptide comprises at least 85% amino acid sequence identity to an amino acid sequence of SEQ ID NO:2 or at least 70% amino acid identity to an amino acid sequence of SEQ ID NO:4 or SEQ ID NO:6; and
(ii) determining the functional effect of the compound on the polypeptide.

34. The method of claim 33, wherein the polypeptide is linked to a solid phase.

35. The method of claim 33, wherein the polypeptide is expressed in a cell or cell membrane.

36. The method of claim 33, wherein the polypeptide has an amino acid sequence of SEQ ID NO:2, SEQ ID NO:4 or SEQ ID NO:6.

37. A method of treating a disease or condition associated with the activity of a PRC17 polypeptide, the method comprising the step of administering to a subject a therapeutically effective amount of a compound identified using the method of claim 33.

38. The method of claim 37, wherein the subject is a human.

39. The method of claim 18, wherein the compound is an antibody.

40. A method of inhibiting proliferation of a cancer cell that expresses a polypeptide comprising at least 85% amino acid sequence identity to an amino acid sequence of SEQ ID NO:2 or at least 70% amino acid identity to an amino acid sequence of SEQ ID NO:4 or SEQ ID NO:6, the method comprising the step of contacting the cancer cell with a therapeutically effective amount of an inhibitor of the polypeptide.

41. The method of claim 40, wherein the polypeptide has an amino acid sequence of SEQ ID NO:2, SEQ ID NO:4 or SEQ ID NO:6.

42. The method of claim 40, wherein the cancer cell is selected from the group consisting of a prostate cancer cell, a breast cancer cancer cell, a lung cancer cell or an ovarian cancer cell.

43. The method of claim 40, wherein the inhibitor is an antibody.


SEQUENCE LISTING (18 sequences)

SEQ ID NO: 1 - DNA, 1964 bp, Homo sapiens
  human PRC17
atggacgtggtagaggtcgcgggcagttggtgggcacaagagcgagaggacatcattatgaaatacgaaaagggacaccgagctgggctgccagaggacaaggggcctaagccttttcgaagctacaacaacaacgtcgatcatttggggattgtacatgagacggagctgcctcctctgactgcgcgggaggcgaagcaaattcggcgggagatcagccgaaagagcaagtgggtggatatgctgggagactgggagaaatacaaaagcagcagaaagctcatagatcgagcgtacaagggaatgcccatgaacatccggggcccgatgtggtcagtcctcctgaacattgaggaaatgaagttgaaaaaccccggaagataccagatcatgaaggagaagggcaagaggtcatctgagcacatccagcgcatcgaccgggacgtaagcgggacattaaggaagcatatattcttcagggatcgatacggaaccaagcagcgggaactactccacatcctcctggcatatgaggagtataacccggaggtgggctactgcagggacctgagccacatcgccgccttgttcctcctctatcttcctgaggaggatgcattctgggcactggtgcagctgctggccagtgagaggcactccctgcagggatttcacagcccaaatggcgggaccgtccaggggctccaagaccaacaggagcatgtggtagccacgtcacaacccaagaccatggggcatcaggacaagaaagatctatgtgggcagtgttccccgttaggctgcctcatccggatattgattgacgggatctctctcgggctcaccctgcgcctgtgggacgtgtatctggtagaaggcgaacaggcgttgatgccgataacaagaatcgcctttaaggttcagcagaagcgcctcacgaagacgtccaggtgtggcccgtgggcacgtttttgcaaccggttcgttgatacctgggccagggatgaggacactgtgctcaagcatcttagggcctctatgaagaaactaacaagaaagcagggggacctgccacccccagccaaacccgagcaagggtcgtcggcatccaggcctgtgccggcttcacgtggcgggaagaccctctgcaagggggacaggcaggcccctccaggcccaccagcccggttcccgcggcccatttggtcagcttccccgccacgggcacctcgttcttccacaccctgtcctggtggggctgtccgggaagacacctaccctgtgggcactcagggtgtgcccagcccggccctggctcagggaggacctcagggttcctggagattcctgcagtggaactccatgccccgcctcccaacggacctggacgtagagggcccttggttccgccattatgatttcagacagagctgctgggtccgtgccatatcccaggaggaccagctggccccctgctggcaggctgaacaccctgcggagcgggtgagatcggctttcgctgcacccagcactgattccgaccagggcacccccttcagagctagggacgaacagccgtgtgctcccacctcagggccttgcctctgcggcctccacttggaaagttctcagttccctccaggcttctagaagcatctgggccagggctcatggctggataatttccctaggcttaacaacccaagcaagcttcgcgtcctcgttttatttttggttaaacttatgaaaatgtattaagaaagagtgcagctcgagagagattcagagatggaacacaccagaccccagatcacaaagccaaccatgcccagcccctcccagcacccccagccccacgaccatcgttctgaattctgacgacaccgtgagcctgcctttgtactttaaactcatggaaggataactaccttcacgttttgaaataaatgtttcctgttgaaatg

SEQ ID NO: 2 - protein, 549 aa, Homo sapiens
  human PRC17
MDVVEVAGSWWAQEREDIIMKYEKGHRAGLPEDKGPKPFRSYNNNVDHLGIVHETELPPLTAREAKQIRREISRKSKWVDMLGDWEKYKSSRKLIDRAYKGMPMNIRGPMWSVLLNIEEMKLKNPGRYQIMKEKGKRSSEHIQRIDRDVSGTLRKHIFFRDRYGTKQRELLHILLAYEEYNPEVGYCRDLSHIAALFLLYLPEEDAFWALVQLLASERHSLQGFHSPNGGTVQGLQDQQEHVVATSQPKTMGHQDKKDLCGQCSPLGCLIRILIDGISLGLTLRLWDVYLVEGEQALMPITRIAFKVQQKRLTKTSRCGPWARFCNRFVDTWARDEDTVLKHLRASMKKLTRKQGDLPPPAKPEQGSSASRPVPASRGGKTLCKGDRQAPPGPPARFPRPIWSASPPRAPRSSTPCPGGAVREDTYPVGTQGVPSPALAQGGPQGSWRFLQWNSMPRLPTDLDVEGPWFRHYDFRQSCWVRAISQEDQLAPCWQAEHPAERVRSAFAAPSTDSDQGTPFRARDEQPCAPTSGPCLCGLHLESSQFPPGF

SEQ ID NO: 3 - DNA, 2146 bp, Homo sapiens
  human PRC17 splice variant 1 (exon 3 expanded)
atggacgtggtagaggtcgcgggcagttggtgggcacaagagcgagaggacatcattatgaaatacgaaaagggacaccgagctgggctgccagaggacaaggggcctaagccttttcgaagctacaacaacaacgtcgatcatttggggattgtacagtcctgccgctcctgggagtcagccccacaggaaggcccttgtcctcccttccctgtgccttctcctgggctgagccctgagctggaaagggacagagccagtcctttctgggggtcggcacccaggctggggccgctccaggccccgtgcagttcctcagctctgcctgggttgccttacagtgagacggagctgcctcctctgactgcgcgggaggcgaagcaaattcggcgggagatcagccgaaagagcaagtgggtgatatgctgggagactgggagaaatacaaaagcagcagaaagctcatagatcgagcgtacaagggaatgcccatgaacatccggggcccgatgtggtcagtcctcctgaacattgaggaaatgaagttgaaaaaccccggaagataccagatcatgaaggagaagggcaagaggtcatctgagcacatccagcgcatcgaccgggacgtaagcgggacattaaggaagcatatattcttcagggatcgatacggaaccaagcagcgggaactactccacatcctcctggcatatgaggagtataacccggaggtgggctactgcagggacctgagccacatcgccgccttgttcctcctctatcttcctgaggaggatgcattctgggcactggtgcagctgctggccagtgagaggcactccctgcagggatttcacagcccaaatggcgggaccgtccaggggctccaagaccaacaggagcatgtggtagccacgtcacaacccaagaccatggggcatcaggacaagaaagatctatgtgggcagtgttccccgttaggctgcctcatccggatattgattgacgggatctctctcgggctcaccctgcgcctgtgggacgtgtatctggtagaaggcgaacaggcgttgatgccgataacaagaatcgcctttaaggttcagcagaagcgcctcacgaagacgtccaggtgtggcccgtgggcacgtttttgcaaccggttcgttgatacctgggccagggatgaggacactgtgctcaagcatcttagggcctctatgaagaaactaacaagaaagcagggggacctgccacccccagccaaacccgagcaagggtcgtcggcatccaggcctgtgccggcttcacgtggcgggaagaccctctgcaagggggacaggcaggcccctccaggcccaccagcccggttcccgcggcccatttggtcagcttccccgccacgggcacctcgttcttccacaccctgtcctggtggggctgtccgggaagacacctaccctgtgggcactcagggtgtgcccagcccggccctggctcagggaggacctcagggttcctggagattcctgcagtggaactccatgccccgcctcccaacggacctggacgtagagggcccttggttccgccattatgatttcagacagagctgctgggtccgtgccatatcccaggaggaccagctggccccctgctggcaggctgaacaccctgcggagcgggtgagatcggctttcgctgcacccagcactgattccgaccagggcacccccttcagagctagggacgaacagccgtgtgctcccacctcagggccttgcctctgcggcctccacttggaaagttctcagttccctccaggcttctagaagcatctgggccagggctcatggctggataatttccctaggcttaacaacccaagcaagcttcgcgtcctcgttttatttttggttaaacttatgaaaatgtattaagaaagagtgcagctcgagagagattcagagatggaacacaccagaccccagatcacaaagccaaccatgcccagcccctcccagcacccccagccccacgaccatcgttctgaattctgacgacaccgtgagcctgcctttgtactttaaactcatggaaggataactaccttcacgttttgaaataaatgtttcctgttgaaatg

SEQ ID NO: 4 - protein, 610 aa, Homo sapiens
  human PRC17 splice variant 1 (exon 3 expanded)
MDVVEVAGSWWAQEREDIIMKYEKGHRAGLPEDKGPKPFRSYNNNVDHLGIVQSCRSWESAPQEGPCPPFPVPSPGLSPELERDRASPFWGSAPRLGPLQAPCSSSALPGLPYSETELPPLTAREAKQIRREISRKSKWVDMLGDWEKYKSSRKLIDRAYKGMPMNIRGPMWSVLLNIEEMKLKNPGRYQIMKEKGKRSSEHIQRIDRDVSGTLRKHIFFRDRYGTKQRELLHILLAYEEYNPEVGYCRDLSHIAALFLLYLPEEDAFWALVQLLASERHSLQGFHSPNGGTVQGLQDQQEHVVATSQPKTMGHQDKKDLCGQCSPLGCLIRILIDGISLGLTLRLWDVYLVEGEQALMPITRIAFKVQQKRLTKTSRCGPWARFCNRFVDTWARDEDTVLKHLRASMKKLTRKQGDLPPPAKPEQGSSASRPVPASRGGKTLCKGDRQAPPGPPARFPRPIWSASPPRAPRSSTPCPGGAVREDTYPVGTQGVPSPALAQGGPQGSWRFLQWNSMPRLPTDLDVEGPWFRHYDFRQSCWVRAISQEDQLAPCWQAEHPAERVRSAFAAPSTDSDQGTPFRARDEQPCAPTSGPCLCGLHLESSQFPPGF

SEQ ID NO: 5 - DNA, 1862 bp, Homo sapiens
  human PRC17 splice variant 2 (exon 10 deleted)
atggacgtggtagaggtcgcgggcagttggtgggcacaagagcgagaggacatcattatgaaatacgaaaagggacaccgagctgggctgccagaggacaaggggcctaagccttttcgaagctacaacaacaacgtcgatcatttggggattgtacatgagacggagctgcctcctctgactgcgcgggaggcgaagcaaattcggcgggagatcagccgaaagagcaagtgggtggatatgctgggagactgggagaaatacaaaagcagcagaaagctcatagatcgagcgtacaagggaatgcccatgaacatccggggcccgatgtggtcagtcctcctgaacattgaggaaatgaagttgaaaaaccccggaagataccagatcatgaaggagaagggcaagaggtcatctgagcacatccagcgcatcgaccgggacgtaagcgggacattaaggaagcatatattcttcagggatcgatacggaaccaagcagcgggaactactccacatcctcctggcatatgaggagtataacccggaggtgggctactgcagggacctgagccacatcgccgccttgttcctcctctatcttcctgaggaggatgcattctgggcactggtgcagctgctggccagtgagaggcactccctgcagggatttcacagcccaaatggcgggaccgtccaggggctccaagaccaacaggagcatgtggtagccacgtcacaacccaagaccatggggcatcagtatctggtagaaggcgaacaggcgttgatgccgataacaagaatcgcctttaaggttcagcagaagcgcctcacgaagacgtccaggtgtggcccgtgggcacgtttttgcaaccggttcgttgatacctgggccagggatgaggacactgtgctcaagcatcttagggcctctatgaagaaactaacaagaaagcagggggacctgccacccccagccaaacccgagcaagggtcgtcggcatccaggcctgtgccggcttcacgtggcgggaagaccctctgcaagggggacaggcaggcccctccaggcccaccagcccggttcccgcggcccatttggtcagcttccccgccacgggcacctcgttcttccacaccctgtcctggtggggctgtccgggaagacacctaccctgtgggcactcagggtgtgcccagcccggccctggctcagggaggacctcagggttcctggagattcctgcagtggaactccatgccccgcctcccaacggacctggacgtagagggcccttggttccgccattatgatttcagacagagctgctgggtccgtgccatatcccaggaggaccagctggccccctgctggcaggctgaacaccctgcggagcgggtgagatcggctttcgctgcacccagcactgattccgaccagggcacccccttcagagctagggacgaacagccgtgtgctcccacctcagggccttgcctctgcggcctccacttggaaagttctcagttccctccaggcttctagaagcatctgggccagggctcatggctggataatttccctaggcttaacaacccaagcaagcttcgcgtcctcgttttatttttggttaaacttatgaaaatgtattaagaaagagtgcagctcgagagagattcagagatggaacacaccagaccccagatcacaaagccaaccatgcccagcccctcccagcacccccagccccacgaccatcgttctgaattctgacgacaccgtgagcctgcctttgtactttaaactcatggaaggataactaccttcacgttttgaaataaatgtttcctgttgaaatg

SEQ ID NO: 6 - protein, 515 aa, Homo sapiens
  human PRC17 splice variant 2 (exon 10 deleted)
MDVVEVAGSWWAQEREDIIMKYEKGHRAGLPEDKGPKPFRSYNNNVDHLGIVHETELPPLTAREAKQIRREISRKSKWVDMLGDWEKYKSSRKLIDRAYKGMPMNIRGPMWSVLLNIEEMKLKNPGRYQIMKEKGKRSSEHIQRIDRDVSGTLRKHIFFRDRYGTKQRELLHILLAYEEYNPEVGYCRDLSHIAALFLLYLPEEDAFWALVQLLASERHSLQGFHSPNGGTVQGLQDQQEHVVATSQPKTMGHQYLVEGEQALMPITRIAFKVQQKRLTKTSRCGPWARFCNRFVDTWARDEDTVLKHLRASMKKLTRKQGDLPPPAKPEQGSSASRPVPASRGGKTLCKGDRQAPPGPPARFPRPIWSASPPRAPRSSTPCPGGAVREDTYPVGTQGVPSPALAQGGPQGSWRFLQWNSMPRLPTDLDVEGPWFRHYDFRQSCWVRAISQEDQLAPCWQAEHPAERVRSAFAAPSTDSDQGTPFRARDEQPCAPTSGPCLCGLHLESSQFPPGF

SEQ ID NO: 7 - DNA, 22 bp, Artificial Sequence
  Description of Artificial Sequenceforward primer for PCR amplification of PRC17 cDNA
ggagggaactgagaactttcca

SEQ ID NO: 8 - DNA, 21 bp, Artificial Sequence
  Description of Artificial Sequencereverse primer for PCR amplification of PRC17 cDNA
cgaacagcagtatgctcccac

SEQ ID NO: 9 - DNA, 20 bp, Artificial Sequence
  Description of Artificial Sequencefluorescent-labeled PCR TaqMan detection probe for PRC17
ncagggccttgcctctgcgn

SEQ ID NO: 10 - DNA, 18 bp, Artificial Sequence
  Description of Artificial Sequenceforward primer for full-length PRC17 splice variant 1 cDNA isolation
ggatatggcacggaccca

SEQ ID NO: 11 - DNA, 19 bp, Artificial Sequence
  Description of Artificial Sequencereverse primer for full-length PRC17 splice variant 1 cDNA isolation
ggacctggacgtagagggc

SEQ ID NO: 12 - DNA, 29 bp, Artificial Sequence
  Description of Artificial SequencePCR TaqMan detection probe for PRC17 splice variant 1
nctgtctgaaatcataatggcggaaccan

SEQ ID NO: 13 - protein, 6 aa, Artificial Sequence
  Description of Artificial Sequence6-His epitope tag
HHHHHH

SEQ ID NO: 14 - protein, 8 aa, Artificial Sequence
  Description of Artificial Sequenceanti- DYKDDDDK epitope tag
DYKDDDDK

SEQ ID NO: 15 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 16 - protein, 18 aa, Artificial Sequence
  Description of Artificial Sequenceunique PRC17 C-terminal peptide
PSTSDQGTPFRARDEQPC

SEQ ID NO: 17 - protein, 786 aa, Homo sapiens
  human TRE-2/USP6
MDMVENADSLQAQERKDILMKYDKGHRAGLPEDKGPEPVGINSSIDRFGILHETELPPVTAREAKKIRREMTRTSKWMEMLGEWETYKHSSKLIDRVYKGIPMNIRGPVWSVLLNIQEIKLKNPGRYQIMKERGKRSSEHIHHIDLDVRTTLRNHVFFRDRYGAKQRELFYILLAYSEYNPEVGYCRDLSHITALFLLYLPEEDAFWALVQLLASERHSLPGFHSPNGGTVQGLQDQQEHVVPKSQPKTMWHQDKEGLCGQCASLGCLLRNLIDGISLGLTLRLWDVYLVEGEQVLMPITSIALKVQQKRLMKTSRCGLWARLRNQFFDTWAMNDDTVLKHLRASTKKLTRKQGDLPPPAKREQGSLAPRPVPASRGGKTLCKGYRQAPPGPPAQFQRPICSASPPWASRFSTPCPGGAVREDTYPVGTQGVPSLALAQGGPQGSWRFLEWKSMPRLPTDLDIGGPWFPHYDFERSCWVRAISQEDQLATCWQAEHCGEVHNKDMSWPEEMSFTANSSKIDRQKVPTEKGATGLSNLGNTCFMNSSIQCVSNTQPLTQYFISGRHLYELNRTNPIGMKGHMAKCYGDLVQELWSGTQKSVAPLKLRRTIAKYAPKFDGFQQQDSQELLAFLLDGLHEDLNRVHEKPYVELKDSDGRPDWEVAAEAWDNHLRRNRSIIVDLFHGQLRSQVKCKTCGHISVRFDPFNFLSLPLPMDSYMDLEITVIKLDGTTPVRYGLRLNMDEKYTGLKKQLRDLCGLNSEQILLAEVHDSNIKISPLHHLQMECSP

SEQ ID NO: 18 - protein, 549 aa, Homo sapiens
  human PRC17.codi
MDVVEVAGSWWAQEREDIIMKYEKGHRAGLPEDKGPKPFRSYNNNVDHLGIVHETELPPLTAREAKQIRREISRKSKWVDMLGDWEKYKSSRKLIDRAYKGMPMNIRGPMWSVLLNIEEMKLKNPGRYQIMKEKGKRSSEHIQRIDRDISGTLRKHMFFRDRYGTKQRELLHILLAYEEYNPEVGYCRDLSHIAALFLLYLPEEDAFWALVQLLASERHSLQGFHSPNGGTVQGLQDQQEHVVATSQSKTMGHQDKKDLCGQCSPLGCLIRILIDGISLGLTLRLWDVYLVEGEQALMPITRIAFKVQQKRLTKTSRCGPWARFCNRFVDTWARDEDTVLKHLRASMKKLTRKQGDLPPPAKPEQGSSASRPVPASRGGKTLCKGDRQAPPGPPARFPRPIWSASPPRAPRSSTPCPGGAVREDTYPVGTQGVPSPALAQGGPQGSWRFLQWNSMPRLPTDLDVEGPWFRHYDFRQSCWVRAISQEDQLAPCWQAEHPAERVRSAFAAPSTDSDQGTPFRARDEQQYAPTSGPCLCGLHLESSQFPPGF